(19) United States
(12) Patent Application Publication
(10) Pub. No.: US 2024/0365738 A1
(43) Pub. Date: Nov. 7, 2024

(54) FACULTATIVE WINTER CAMELINA PLANTS

(71) Applicant: Regents of the University of Minnesota, Minneapolis, MN (US)

(72) Inventors: James Allan ANDERSON, Woodbury, MN (US); Matthew Ames OTT, Saint Paul, MN (US); Ratan CHOPRA, Saint Louis, MO (US); Katherine FRELS, Lincoln, NE (US); Michael David MARKS, Trempealeau, WI (US)

(73) Assignee: Regents of the University of Minnesota, Minneapolis, MN (US)

(21) Appl. No.: 18/654,357

(22) Filed: May 3, 2024

Related U.S. Application Data
(60) Provisional application No. 63/499,907, filed on May 3, 2023.

(51) Int. Cl. A01H 6/20; A01H 5/10

(52) U.S. Cl. CPC A01H 6/20; A01H 5/10

(57) ABSTRACT

The present disclosure generally relates to Camelina sativa plants exhibiting facultative wintering. Provided herein are methods and compositions for producing plants exhibiting facultative wintering. Also provided herein are exemplary plants exhibiting this trait, including, for example, Camelina sativa line ‘CMN2207’.

### CROSS-REFERENCE TO RELATED APPLICATION

This application claims the benefit of U.S. Provisional Application No. 63/499,907, filed May 3, 2023, which is hereby incorporated by reference in its entirety.

### REFERENCE TO AN ELECTRONIC SEQUENCE LISTING

The contents of the electronic sequence listing (144832001200SEQLIST.xml; Size: 3,766 bytes; and Date of Creation: Apr. 19, 2024) is herein incorporated by reference in its entirety.

### FIELD

The present disclosure generally relates to Camelina sativa plants exhibiting facultative wintering. Provided herein are methods and compositions for producing plants exhibiting facultative wintering. Also provided herein are exemplary plants exhibiting this trait, including, for example, Camelina sativa line ‘CMN2207’.

### BACKGROUND

Camelina sativa is being developed as a winter oilseed cover crop. Early flowering and maturity are desired traits in Camelina sativa to allow for relay planting or seeding of a summer annual following Camelina sativa harvest.

There is unprecedented global demand for biofuels and feed byproducts as consumers seek out fossil fuel alternatives to mitigate climate change and energy price volatility. The energy producer Exxon Mobil has partnered with Global Clean Energy to purchase 220 million gallons of oil from spring camelina in the western US (Sustainable Oils Camelina (susoils.com) 2022). In the Upper Midwest winter camelina is being developed as a new cover/cash crop that can be planted in the fall and harvested in spring on the same lands used for traditional summer crop such as that undergoing the maize to soybean rotation on much the Midwestern farm acreage. When planted as a winter annual oilseed cover crop, Camelina sativa (L. Crantz) should be able to ease some of the supply shortage issues for both the energy and feed sectors. Fall planted/spring harvested camelina as an oilseed cover crop also provides important ecosystem services, such as reducing nitrate leaching and providing supplemental nutrition for pollinators, further promoting the crop as a potential solution to several complex challenges (Weyers et al., 2019; Eberle et al., 2015).

Camelina sativa has been shown to be descended from Camelina macrocarpa, with a region of origin spanning the Mediterranean and Eastern Europe (Brock et al., 2018; Chaudhary et al., 2020). It remains unclear exactly when Camelina sativa began to be actively cultivated rather than being tolerated as a weed in cultivated flax (Brock et al., 2018), but its use as a crop can be documented as far back as the early Iron Age (˜1200 BCE) (Zohary et al., 2012). Two cytotypes exist for allohexaploid Camelina sativa—either n=6+7+7 (20) or n=6+6+7 (19) (Hotton et al., 2020). The genome size was estimated to be 785 Mb, which is relatively small for a hexaploid species (Kagale et al., 2014). Camelina is closely related to Arabidopsis thaliana as both are in Brassicacea lineage group I (Huang et al., 2016). Similar to Arabidopsis, camelina is largely a self-fertilized species. In the Camelina sativa germplasm within the US's National Genetic Resources Program, only eight of the 41 accessions exhibited the winter biotype, while the rest exhibited the spring biotype and are not winter-hardy (Hotton et al., 2020). This leaves plant breeders focused on winter types for their value as a winter cover crop with a narrow genetic base.

To increase genetic diversity for key traits in winter camelina, one strategy is to cross-pollinate winter and spring types, and either select for winter types in the field if feasible, or backcross as necessary to recover the winter growth habit while introducing impactful alleles. Identifying genetic markers that can predict the winter/spring growth habits in Camelina sativa would save time otherwise spent waiting for populations to phenotypically segregate for this trait before selection and desirable allele stacking could take place in a breeding program.

Thus, there is a need to develop Camelina sativa plants with alternative growth habits, such as Camelina sativa plants exhibiting facultative wintering.

### BRIEF SUMMARY

In order to meet these needs, the present disclosure is directed to Camelina sativa plants exhibiting facultative wintering, methods and compositions for producing plants exhibiting facultative wintering, and exemplary plants exhibiting this trait, including, for example, Camelina sativa line ‘CMN2207’.

Certain aspects of the present disclosure relate to a Camelina sativa seed designated as ‘CMN2207’, representative sample of seed having been deposited under ATCC Accession Number X1.

Certain aspects of the present disclosure relate to a Camelina sativa plant produced by growing the seed of any of the preceding embodiments.

Certain aspects of the present disclosure relate to a plant part from the plant of any of the preceding embodiments, wherein said part is a leaf, a flower, an ovule, a pollen grain, a seed pod, a seed, a fruit, a cell, or a portion thereof. In some embodiments, the plant part is a seed.

Certain aspects of the present disclosure relate to a Camelina sativa plant having all the physiological and morphological characteristics of the Camelina sativa plant of any of the preceding embodiments.

Certain aspects of the present disclosure relate to a plant part from the plant of any of the preceding embodiments, wherein said part is a leaf, an ovule, a pollen grain, a seed pod, a seed, a fruit, a cell, or a portion thereof. In some embodiments, the part is a seed.

Certain aspects of the present disclosure relate to a F1 hybrid Camelina sativa plant having ‘CMN2207’ as a parent where ‘CMN2207’ is grown from the seed of any of the preceding embodiments.

Certain aspects of the present disclosure relate to a pollen grain or an ovule of the plant of any of the preceding embodiments.

Certain aspects of the present disclosure relate to a tissue or cell culture produced from protoplasts or cells from the plant of any of the preceding embodiments, wherein said cells or protoplasts are produced from a plant part selected from the group consisting of root, root tip, meristematic cell, stem, hypocotyl, petiole, cotyledon, leaf, flower, anther, pollen, pistil, embryo, seed, and fruit.

Certain aspects of the present disclosure relate to a Camelina sativa plant regenerated from the tissue culture of any of the preceding embodiments, wherein the plant has all of the morphological and physiological characteristics of a Camelina sativa plant produced by growing Camelina sativa seed designated as ‘CMN2207’, representative sample of seed having been deposited under ATCC Accession Number X1.

Certain aspects of the present disclosure relate to a method of making Camelina sativa seeds including crossing the plant of any of the preceding embodiments with itself or with another Camelina sativa plant and harvesting seed therefrom. In some embodiments, the method further includes: (a) selecting one or more of the F1 progeny for plants that have at least one desired trait to produce selected progeny plants; (b) crossing the selected progeny plants with ‘CMN2207’ to produce backcross progeny plants, a representative sample of seed of ‘CMN2207’ having been deposited under ATCC Accession Number X1; (c) selecting for backcross progeny plants that have the desired trait and all of the physiological and morphological characteristics of ‘CMN2207’ when grown in the same environmental conditions to produce selected backcross progeny plants; and (d) repeating steps (b) and (c) three or more times in succession to produce selected fourth or higher backcross progeny plants that include the desired trait and all of the physiological and morphological characteristics of ‘CMN2207’ when grown in the same environmental conditions. In some embodiments, the at least one desired trait is selected from the group consisting of: ability to flower absent vernalization; winter hardiness; reaching maturity earlier than control fully winter Camelina accessions when grown under the same conditions; resistance to Ceutorhynchus erysimi (weevils); seed area; seed mass; seed yield; and plant height. In some embodiments, a plant is deemed to mature earlier than the control plant when a plot of said plant reaches 50% maturity in fewer days than a plot of the control plant.

Certain aspects of the present disclosure relate to a method of producing a seed of ‘CMN2207’-derived Camelina sativa plant including: a) crossing a Camelina sativa plant designated as ‘CMN2207’ with a second Camelina sativa plant, whereby seed of a ‘CMN2207’-derived Camelina sativa plant forms. In some embodiments, the method further includes: b) crossing a plant grown from ‘CMN2207’-derived Camelina sativa seed with itself or a second Camelina sativa plant to yield additional ‘CMN2207’-derived Camelina sativa seed; c) growing the additional ‘CMN2207’-derived Camelina sativa seed of step (b) to yield additional ‘CMN2207’-derived Camelina sativa plants; and d) repeating the crossing and growing of steps (b) and (c) for an additional 3-10 generations to generate further ‘CMN2207’-derived Camelina sativa plants.

Certain aspects of the present disclosure relate to a method of producing a Camelina sativa seed including growing the plant of any of the preceding embodiments until it produces at least one seed pod, and harvesting the seeds.

Certain aspects of the present disclosure relate to a method of producing a seed of ‘CMN2207’-derived Camelina sativa including: a) crossing a Camelina sativa plant designated as ‘CMN2207’, representative sample of seed having been deposited under ATCC Accession Number X1, with itself or with a second Camelina sativa plant, whereby seed of a ‘CMN2207’-derived Camelina sativa plant forms. In some embodiments, the method further includes: b) crossing a plant grown from ‘CMN2207’-derived Camelina sativa seed with itself or with a second Camelina sativa plant to yield additional ‘CMN2207’-derived Camelina sativa seed; c) growing the additional ‘CMN2207’-derived Camelina sativa seed of step (b) to yield additional ‘CMN2207’-derived Camelina sativa plants; and d) repeating the crossing and growing of steps (b) and (c) for an additional 3-10 generations to generate further ‘CMN2207’-derived Camelina sativa plants.

Certain aspects of the present disclosure relate to a method of vegetatively propagating a plant of Camelina sativa ‘CMN2207’ including the steps of: (a) collecting tissue capable of being propagated from a plant of Camelina sativa ‘CMN2207’, representative seed of said Camelina sativa variety having been deposited under ATCC Accession Number X1; and (b) producing a rooted plant from said tissue.

Certain aspects of the present disclosure relate to a Camelina sativa plant produced by the method of any of the preceding embodiments, wherein said plant reaches maturity earlier than control fully winter Camelina accessions when grown under the same conditions, wherein said plant flowers absent vernalization, and wherein said plant incudes a subfunctional FLC allele on chromosome 20.

Certain aspects of the present disclosure relate to a plant part from the plant of any of the preceding embodiments, wherein the part is a leaf, an ovule, a pollen grain, a seed pod, a seed, a fruit, a cell, or a portion thereof.

Certain aspects of the present disclosure relate to a Camelina sativa plant that 1) flowers and reaches maturity earlier than a control winter Camelina accession when grown under the same conditions, 2) flowers and reaches maturity later than a control spring Camelina accession when grown under the same conditions without vernalization in a non-freezing environment, 3) can survive in a freezing environment after transitioning to a floral meristem, and 4) is able to flower absent vernalization. In some embodiments, the plant further exhibits at least one trait selected from the group consisting of: resistance to Ceutorhynchus erysimi (weevils); a seed area in the range of about 1.2 mm² to about 1.4 mm²; a seed mass in the range of about 0.7 grams per thousand seeds to about 1 gram per thousand seeds; a seed yield in the range of about 1100 kg/ha to about 1300 kh/ha; and a plant height in the range of about 57 cm to about 65 cm. In some embodiments, the plant includes a non-functional FLOWERING LOCUS C gene on chromosome 20. In some embodiments, representative seeds of such plant have been deposited under ATCC Accession Number X1.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows Julian days for camelina lines to reach 50% plot flowering in St. Paul, MN, 2022. n=4.

FIG. 2 shows Julian days for camelina lines to reach 50% plot flowering in Morris, MN, 2022. n=4.

FIG. 3 shows days after planting for camelina lines to reach 50% plot flowering in American Falls, ID, 2022. n=4.

FIG. 4 shows Julian days for camelina lines to reach 50% plot maturity in St. Paul, MN, 2022. n=4.

FIG. 5 shows days after planting for camelina lines to reach 50% plot maturity in American Falls, ID, 2022. n=4.

FIG. 6 shows seed yield of camelina lines in St. Paul, MN in 2022. n=4.

FIG. 7 shows seed yield of camelina lines in Morris, MN in 2022. n=4.

FIG. 8 shows seed yield of camelina lines in American Falls, ID in 2022. n=4.

FIG. 9 shows flowering and maturity results from a camelina yield trial planted on 14 Sep. 2021 in in St. Paul, MN in a randomized complete block design. PI 650163-1 and PI 650168 are the parents of CMN2207 (F₄:F₆). Capital letters refer to mean maturity comparisons and lower-case letters refer to mean flowering comparisons. Values over bars with the same letter are not significantly different. n=4. LSD flowering=3.26. LSD maturity=0.58.

FIG. 10 shows 50% plot flowering and results from a camelina yield trial planted on 16 Sep. 2021 in Morris, MN in a randomized complete block design. PI 650163-1 and PI 650168 are the parents of CMN2207 (F₄:F₆). Values over bars with the same letter are not significantly different. n=4. LSD=1.29.

FIG. 11 shows Camelina yield trial results from St. Paul, MN. Yield trial planted on 14 Sep. 2021 in a randomized complete block design. PI 650163-1 and PI 650168 are the parents of CMN2207 (F₄:F₆). Values over bars with the same letter are not significantly different. n=4. LSD=394.22.

FIG. 12 shows Camelina yield trial results from Morris, MN. Yield trial planted on 16 Sep. 2021 in a randomized complete block design. PI 650163-1 and PI 650168 are the parents of CMN2207 (F₄:F₆). Values over bars with the same letter are not significantly different. n=4. LSD=290.86.

FIG. 13 shows seed area results from camelina yield trials planted in mid-September in St. Paul, and Morris, MN in a randomized complete block design. Seeds were analyzed with a Marvin ProLine Seed Analyzer (Marvitech GmbH, Wittenburg, Germany). PI 650163-1 and PI 650168 are the parents of CMN2207 (F₄:F₆). Values over bars with the same letter are not significantly different. n=8. LSD=0.066.

FIG. 14 shows thousand seed weight results from camelina yield trials planted in mid-September in St. Paul, and Morris, MN in a randomized complete block design. Seeds were analyzed with a Marvin ProLine Seed Analyzer (Marvitech GmbH, Wittenburg, Germany). PI 650163-1 and PI 650168 are the parents of CMN2207 (F₄:F₆). A parametric test did not meet assumptions of ANOVA, so a non-parametric Kruskal-Wallis test was used in conjunction with a Dunn's test for mean comparisons. (P<0.01). n=8.

FIG. 15 shows Shephard's purse weevil larvae injury (Ceutorhynchus erysimi) on Camelina from yield trial in St. Paul, MN. Yield trial planted on 14 Sep. 2021 in a randomized complete block design. PI 650163-1 and PI 650168 are the parents of CMN2207 (F₄:F₆). Values over bars with the same letter are not significantly different. There was no incidence of this weevil larvae at the Morris site. n=4. LSD=9.56.

FIG. 16 shows Camelina plant height from yield trial in St. Paul, MN. Yield trial planted on 14 Sep. 2021 in a randomized complete block design. PI 650163-1 and PI 650168 are the parents of CMN2207 (F₄:F₆). Values over bars with the same letter are not significantly different. n=4. LSD=3.43.

FIG. 17 shows Camelina plant height from yield trial in Morris, MN. Yield trial planted on 16 Sep. 2021 in a randomized complete block design. PI 650163-1 and PI 650168 are the parents of CMN2207 (F₄:F₆). Values over bars with the same letter are not significantly different. n=4. LSD=3.43.

FIG. 18 shows QTL mapping results obtained with QTLseqr and developed using bulked segregant analysis on an F₂ population of a cross between Ames 33292 (Joelle) and P1650163-1. One region on chromosome 20 in Camelina sativa L. Crantz is a primary regulator of flowering time. The FLOWERING LOCUS C gene is within this region.

FIG. 19 shows Kompetitive Allele-Specific PCR (KASP) genotyping results that differentiate winter from spring growth habit accessions of Camelina sativa using the previously identified single basepair INDEL in exon 5 of the FLOWERING LOCUS C gene on chromosome 20 (Anderson et al., 2018).

FIG. 20 shows Camelina shoot height observed in a greenhouse 34 days after planting on Feb. 19, 2016 with no vernalization. Experimental design was a randomized complete block and n=3. The greenhouse temperature was maintained near 20° C. and was supplemented with halogen lighting to ensure 16 h light was available each day.

FIG. 21 shows a photograph of CMN2207 plants (at right; flowering) and Joelle plants (at left) growing in a field in Morris, Minnesota, USA.

FIG. 22 shows a photograph of a side view of a potted CMN2207 plant at flowering stage. The pictured plant was grown in a greenhouse in St. Paul, Minnesota, USA.

### DETAILED DESCRIPTION

### General Terms

The terminology used herein is for the purpose of describing particular embodiments and is not intended to be limiting.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

The term “and/of” as used herein a phrase such as “A and/or B” is intended to include both A and B; A or B; A (alone); and B (alone). Likewise, the term “and/of” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that aspects and embodiments of the present disclosure described herein include “comprising,” “consisting,” and “consisting essentially of” aspects and embodiments.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### Overview

The following description is presented to enable a person of ordinary skill in the art to make and use the various embodiments. Descriptions of specific devices, techniques, methods, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown.

The present disclosure generally relates to Camelina sativa plants exhibiting facultative wintering. Provided herein are methods and compositions for producing plants exhibiting facultative wintering. Also provided herein are exemplary plants exhibiting this trait, including, for example, Camelina sativa line ‘CMN2207’.

In particular, the present disclosure is based, at least in part, on Applicant's discovery and development of a facultative wintering trait in Camelina sativa plants. An exemplary variety exhibiting this trait is Camelina sativa line ‘CMN2207’.

Accordingly, the present disclosure provides, for example, methods of producing plants exhibiting the facultative wintering trait, plants and parts thereof that exhibit the facultative wintering trait, and plants and parts thereof of Camelina sativa line ‘CMN2207’ which exhibits the facultative wintering trait.

Camelina sativa Plants and Parts Thereof

The present disclosure generally relates to Camelina sativa plants and parts thereof. Plants of the present disclosure may include any plant or part of a plant at any stage of development, including seeds, suspension cultures, plant cells, embryos, meristematic regions, callus tissue, leaves, roots, contractile roots, shoots, stems, inflorescence, flowers, flower buds, siliques, gametophytes, sporophytes, pollen, microspores, and progeny thereof. Also included are cuttings, and cell or tissue cultures. As used in conjunction with the present disclosure, plant tissues may include, for example, whole plants, plant cells, plant organs, e.g., leaves, stems, roots, contractile roots, meristems, plant seeds, stems, inflorescence, flowers, flower buds, siliques, protoplasts, callus, cell cultures, and any groups of plant cells organized into structural and/or functional units.

In certain aspects of the present disclosure, a Camelina sativa plant can be described according to its seasonal growing cycle and/or growth habit. Camelina sativa plants of the present disclosure can be described as, for example, a spring-type, a winter-type, or a facultative winter-type accession, biotype, cultivar, line, plant, or variety.

In certain aspects of the present disclosure, a Camelina sativa plant exhibits winter hardiness. Exemplary attributes of a Camelina sativa plant that can exhibit winter hardiness include, for example, the ability to survive in a freezing environment after transitioning to a floral meristem, and the ability to survive through the winter in, for example, the states of Idaho and Minnesota, USA, or in growing environments with temperatures similar to typical winter temperatures in the states of Idaho or Minnesota, USA.

In some aspects, a Camelina sativa plant may be described as spring or spring-type. The meanings of “spring” and “spring-type” Camelina sativa plants are known in the art. Spring-type Camelina sativa plants may exhibit various characteristics, such as, for example, lack of winter hardiness and not requiring vernalization prior to, for example, presence of reproductive stems or “bolting”, elongation of the main stem, inflorescence emergence, flowering, and/or fruit development. In some embodiments, a spring-type Camelina sativa plant does not survive freezing environments after transitioning to a floral meristem. In some embodiments, a spring-type Camelina sativa plant has an allele of a flowering time pathway gene that differentiates it from facultative wintering-type Camelina sativa plants. Exemplary spring-type Camelina sativa cultivars include ‘DH55’, ‘CO46’, ‘CN 113704-1’, ‘CN 113704-2’, ‘CN 113704-3’, ‘CN 113746-1’, ‘CN 113746-2’, ‘CN 113746-3’, and ‘CN 113746-4’.

In some aspects, a Camelina sativa plant may be described as winter-type, winter, or fully winter. The meanings of “winter-type”, “winter”, and “fully winter” Camelina sativa plants are known in the art. Winter-type Camelina sativa plants may exhibit various characteristics, such as, for example, winter hardiness, delayed flowering when compared to a spring-type plant, requiring vernalization prior to, for example, presence of reproductive stems or “bolting”, elongation of the main stem, inflorescence emergence, flowering, and/or fruit development, and having a functional allele of the FLOWERING LOCUS C gene on chromosome 20. Exemplary winter-type Camelina sativa cultivars include ‘Joelle’ (‘Ames 33292’), ‘PI650168’, ‘CN113657’, ‘CN113660’, ‘CN113662’, ‘CN113691’, ‘CN113691-1’, ‘CN113691-2’, ‘CN113691-3’, ‘IHAR247046’, ‘IHAR500033’, ‘IHAR502792’, ‘IHAR502792-1’, ‘IHAR502792-2’, ‘IHAR502792-3’, ‘Ames 33292-1’, ‘Ames 33292-2’, ‘Ames 33292-3’, ‘PI311736’, ‘PI650143’, ‘PI650152’, ‘PI650155’, ‘PI650155-1’, ‘PI650155-2’, ‘PI650155-3’, ‘PI650157-1’, ‘PI650157-2’, ‘PI650157-3’, ‘P1650158’, ‘P1650158-1’, ‘P1650158-2’, and ‘P1650158-3’.

In some aspects, a Camelina sativa plant may be described as facultative winter-type. Facultative winter-type Camelina sativa plants of the present disclosure exhibit the facultative wintering trait. Facultative winter-type Camelina sativa plants and the facultative wintering trait are further described herein.

In certain aspects of the present disclosure, one or more growth stages of Camelina sativa plants may be measured. Growth stages of Camelina sativa plants may be measured by one or more exemplary metrics, including, for example, according to the phenological growth stages of Camelina sativa according to the extended BBCH scale as defined by Martinelli & Galasso (Martinelli & Galasso (2011) Annals of Applied Biology, 158(1)), including, for example, by reference to any of the principal growth stages, two-digit BBCH codes, and three-digit BBCH codes defined therein.

Certain aspects of the present disclosure relate to measuring and/or determining the status of maturity of Camelina sativa plants. Maturity may be measured by one or more exemplary metrics, including, for example, according to the phenological growth stages of Camelina sativa according to the extended BBCH scale as defined by Martinelli & Galasso (Martinelli & Galasso (2011) Annals of Applied Biology, 158(1)), including, for example, by reference to any of principal growth stages 6-8, two-digit BBCH codes 67-89, and three-digit BBCH codes 607-809. Exemplary metrics for measuring maturity include the timing in days of one or more of the following: flowering finishing (majority of petals fallen or dry), end of flowering (fruit set visible), 10% of the siliques having reached the final size, 20% of the siliques having reached the final size, 30% of the siliques having reached the final size, 40% of the siliques having reached the final size, 50% of the siliques having reached the final size, 60% of the siliques having reached the final size, 70% of the siliques having reached the final size, 80% of the siliques having reached the final size, 90% of the siliques having reached the final size, almost all of the siliques having reached the final size, 10% of the siliques being ripe, 20% of the siliques being ripe, 30% of the siliques being ripe, 40% of the siliques being ripe, 50% of the siliques being ripe, 60% of the siliques being ripe, 70% of the siliques being ripe, 80% of the siliques being ripe, 90% of the siliques being ripe, and siliques being ripe for harvest (nearly all of the siliques being ripe). Ripening may be considered to start when, for example, siliques begin to yellow. Siliques may be considered fully ripe when, for example, they appear completely dry and are crunchy to the touch if squeezed, when the seeds inside are deep yellow-orange in color, when the seeds are hard if pressed between one's nails, and/or when the seeds are completely detached from the placenta.

Certain aspects of the present disclosure relate to measuring and/or determining the status of flowering of Camelina sativa plants. Flowering may be measured by one or more exemplary metrics, including, for example, according to the phenological growth stages of Camelina sativa according to the extended BBCH scale as defined by Martinelli & Galasso (Martinelli & Galasso (2011) Annals of Applied Biology, 158(1)), including, for example, by reference to any of principal growth stages 5-6, two-digit BBCH codes 50-69, and three-digit BBCH codes 500-609. Exemplary metrics for measuring flowering include the timing in days of one or more of the following: the inflorescence being present but still enclosed by leaves; the inflorescence being visible from above; individual flower buds being visible but still closed; the first petals being visible outside the sepals but all flowers still being closed; the first flowers opening; 10% of flowers being open (main raceme elongating); 20% of flowers being open; 30% of flowers being open (first petals may be fallen or dry); 40% of flowers being open; 50% of flowers being open (full flowering); flowering finishing (majority of petals fallen or dry), and the end of flowering (fruit set visible). The percentage of flowers being open can be measured, for example, by calculating the percentage of open flowers on the main stem. Calculating the percentage of flowers being open can include, for example, dissecting the inflorescence and counting all of the flower buds present therein, and/or counting the developing siliques that did not attain the final size as flowers.

Camelina sativa Plants Having the Facultative Wintering Trait

Certain aspects of the present disclosure relate to Camelina sativa plants exhibiting facultative wintering. Camelina sativa plants exhibiting facultative wintering may be described as, for example, facultative winter-type, facultative winter, and/or exhibiting facultative wintering. Facultative wintering is a term generally known in the art.

A Camelina sativa plant exhibiting the facultative wintering trait may include, for example, one or more of the following characteristics: 1) flowering and/or reaching maturity earlier than a control winter Camelina accession when grown under the same conditions, 2) flowering and/or reaching maturity later than a control spring Camelina accession when grown under the same conditions without vernalization in a non-freezing environment, 3) ability to survive in a freezing environment after transitioning to a floral meristem, and/or 4) ability to flower absent vernalization.

In some embodiments, facultative wintering in Camelina sativa can include, for example, winter hardiness, flowering and/or reaching maturity earlier than winter-type Camelina sativa plants, and flowering and/or reaching maturity later than spring-type Camelina sativa plants. In some embodiments, a Camelina sativa plant can be considered facultative winter-type when, for example, it exhibits winter hardiness and does not require vernalization prior to, for example, presence of reproductive stems or “bolting”, elongation of the main stem, inflorescence emergence, flowering, and/or fruit development. In some embodiments, a Camelina sativa plant can be considered facultative winter-type when, for example, it can flower and reach maturity earlier than a winter-type Camelina plant when grown under the same conditions, can flower and reach maturity later than a spring-type Camelina plant when grown under the same conditions without vernalization in a non-freezing environment, can survive in a freezing environment after transitioning to a floral meristem, and is able to flower absent vernalization.

In some embodiments, a facultative winter-type Camelina sativa plant has a subfunctional or non-functional allele of the FLOWERING LOCUS C gene on chromosome 20. In some embodiments, a facultative winter-type Camelina sativa plant has a FLOWERING LOCUS C gene on chromosome 20 with a frameshift mutation in exon 5. In some embodiments, a facultative winter-type Camelina sativa plant has a FLOWERING LOCUS C gene on chromosome 20 with a frameshift mutation at base pair (bp) position 4,195,043. In some embodiments, a facultative winter-type Camelina sativa plant has a FLOWERING LOCUS C gene on chromosome 20 with a single nucleotide variant (SNV) mutation in exon 5. In some embodiments, a facultative winter-type Camelina sativa plant has a FLOWERING LOCUS C gene on chromosome 20 with a single nucleotide variant (SNV) mutation at base pair (bp) position 4,195,043.

Exemplary Camelina sativa plants that exhibit facultative wintering include lines ‘CMN2207’ and ‘P1650163-1’. In some embodiments, ‘CMN2207’ exhibits improved traits compared to ‘PI650163-1’. In some embodiments, the improved traits can include, for example, seed area, seed mass, and plant height.

In certain aspects of the present disclosure, a Camelina sativa plant is compared to one or more a control plants. In some embodiments, a control plant may be, for example, a control Camelina sativa plant. Exemplary control Camelina sativa plants may include a winter-type Camelina sativa plant (such as, for example, one or more of the following accessions: ‘Joelle’ (‘Ames 33292’), ‘PI650168’, ‘CN113660’, ‘CN113662’, ‘CN113691’, ‘CN113691-1’, ‘CN113691-2’, ‘CN113691-3’, ‘IHAR247046’, ‘IHAR502792’, ‘IHAR502792-1’, ‘IHAR502792-2’, ‘IHAR502792-3’, ‘Ames 33292-1’, ‘Ames 33292-2’, ‘Ames 33292-3’, ‘PI311736’, ‘PI650143’, ‘PI650155’, ‘PI650155-1’, ‘PI650155-2’, ‘PI650155-3’, ‘PI650157-1’, ‘PI650157-2’, ‘PI650157-3’, ‘PI650158’, ‘P1650158-1’, ‘P1650158-2’, and ‘P1650158-3’) or a spring-type or spring Camelina sativa plant (such as, for example, one or more of the following accessions: ‘DH55’, ‘CO46’, ‘CN 113704-1’, ‘CN 113704-2’, ‘CN 113704-3’, ‘CN 113746-1’, ‘CN 113746-2’, ‘CN 113746-3’, and ‘CN 113746-4’).

In some embodiments, Camelina sativa plants of the present disclosure and a control Camelina sativa plant are grown under the same or substantially the same conditions. In some embodiments, the same conditions may include growth in, for example, a greenhouse; a plant growth chamber with 6400 k white fluorescent lamps and 16 h/8 h day/night cycles; a 4 degree Celsius cold room; a residence comprising a south-facing window and white fluorescent lighting; 5.08 cm diameter×17.8 cm length Deepots™ in a 61×30.5×17.1 cm pot rack in a growth chamber with 6400 k white fluorescent lighting 16 h/8 h day/night cycles; a field in St. Paul, MN, USA after planting of head rows in, for example, September using, for example, a Planet Jr. cone-seeder with a custom-made scoop with approximately 100 seeds per 1.2 meter head row; a field in Morris, MN, USA planted in, for example, September.

In some embodiments, facultative wintering in Camelina sativa can include, for example, early maturation. In some embodiments, early maturation includes maturing earlier than a control plant. In some embodiments, a plant is deemed to mature earlier than a control plant when, for example, the plant or plot of the plant reaches 50% maturity in fewer days than a control plant or plot of the control plant, and the plants and/or plots of the plants are grown in the same conditions. In some embodiments, a plant or plot of said plant reaches 50% maturity in, for example, about 1-10, about 11-20, about 21-30, about 31-40, about 41-50, about 51-60, about 61-70, about 71-80, about 81-90, or about 91-100 fewer days than the control plant or plot of the control plant. In some embodiments, the control plant is a winter-type Camelina sativa plant. In some embodiments, the control plant is one of the following winter-type Camelina sativa accessions: ‘Joelle’ (‘Ames 33292’), ‘PI650168’, ‘CN113660’, ‘CN113662’, ‘CN113691’, ‘CN113691-1’, ‘CN113691-2’, ‘CN113691-3’, ‘IHAR247046’, ‘IHAR502792’, ‘IHAR502792-1’, ‘IHAR502792-2’, ‘IHAR502792-3’, ‘Ames 33292-1’, ‘Ames 33292-2’, ‘Ames 33292-3’, ‘PI311736’, ‘PI650143’, ‘PI650155’, ‘PI650155-1’, ‘PI650155-2’, ‘PI650155-3’, ‘PI650157-1’, ‘PI650157-2’, ‘PI650157-3’, ‘PI650158’, ‘P1650158-1’, ‘P1650158-2’, and ‘P1650158-3’. In some embodiments, the control plant is ‘PI650168’.

In some embodiments, facultative wintering in Camelina sativa can include, for example, early flowering. In some embodiments, early flowering includes flowering earlier than a control plant. In some embodiments, a plant is deemed to flower earlier than a control plant when, for example, a plant or plot of the plant reaches 50% flowering in fewer days than the control plant or plot of the control plant and the plants and/or plots of the plants are grown in the same conditions. In some embodiments, a plant or plot of said plant reaches 50% flowering in, for example, about 1-10, about 11-20, about 21-30, about 31-40, about 41-50, about 51-60, about 61-70, about 71-80, about 81-90, or about 91-100 fewer days than the control plant or plot of the control plant. In some embodiments, the control plant is a winter-type Camelina sativa plant. In some embodiments, the control plant is one of the following winter-type Camelina sativa accessions: ‘Joelle’ (‘Ames 33292’), ‘PI650168’, ‘CN113660’, ‘CN113662’, ‘CN113691’, ‘CN113691-1’, ‘CN113691-2’, ‘CN113691-3’, ‘IHAR247046’, ‘IHAR502792’, ‘IHAR502792-1’, ‘IHAR502792-2’, ‘IHAR502792-3’, ‘Ames 33292-1’, ‘Ames 33292-2’, ‘Ames 33292-3’, ‘PI311736’, ‘PI650143’, ‘PI650155’, ‘PI650155-1’, ‘PI650155-2’, ‘PI650155-3’, ‘PI650157-1’, ‘PI650157-2’, ‘PI650157-3’, ‘PI650158’, ‘P1650158-1’, ‘P1650158-2’, and ‘P1650158-3’. In some embodiments, the control plant is ‘PI650168’. In some embodiments, the control plant is ‘Joelle’.

In some embodiments, facultative wintering in Camelina sativa can include, for example, reaching 50% flowering and 50% maturity in fewer days than a plot of ‘PI650168’ Camelina sativa plants when grown under the same conditions. In some embodiments, facultative wintering in Camelina sativa can include, for example, exhibiting winter hardiness and having a non-functional or subfunctional allele of the FLOWERING LOCUS C gene on chromosome 20.

In some embodiments, plants are grown with vernalization. In some embodiments, vernalization includes, for example, growth in 4° C. In some embodiments, the duration of vernalization may be, for example, less than 1 day, about 1 day, about 1 day to about 1 week, about 1-2 weeks, about 2-3 weeks, about 3 weeks, about 3-4 weeks, about 4-5 weeks, about 5-6 weeks, about 6-7 weeks, about 7-8 weeks, about 8-9 weeks, about 9-10 weeks or more than about 10 weeks.

In some embodiments, plants are grown absent vernalization. In some embodiments, plants grown absent vernalization are grown in conditions including, for example, growth in 16 hour/8 hour day/night cycles at about 20° C., growth in a field, and growth at above about 4° C. for at least about 10-20 days, about 20-30 days, about 30-40 days, about 40-50 days, about 50-60 days, about 60-70 days, about 70-80 days, about 80-90 days, about 90-100 days, about 100-110 days, about 110-120 days, about 120-130 days, about 130-140 days, about 140-150 days, about 150-160 days, about 160-170 days, about 170-180 days, about 180-190 days, about 190-200 days, about 200-210 days, about 210-220 days, about 220-230 days, about 230-240 days, about 240-250 days, about 260-270 days, about 270-280 days, about 280-290 days, about 290-300 days, about 300-310 days, about 310-320 days, about 320-330 days, about 330-340 days, about 340-350 days, about 360-370 days, about 370-380 days, about 380-390 days, about 390-300 days, about 400-410 days, about 410-420 days, about 420-430 days, about 430-440 days, about 440-450 days, about 460-470 days, about 470-480 days, about 480-490 days, about 490-400 days, about 500-510 days, about 510-520 days, about 520-530 days, about 530-540 days, about 540-550 days, about 560-570 days, about 570-580 days, about 580-590 days, about 590-500 days, about 600-610 days, about 610-620 days, about 620-630 days, about 630-640 days, about 640-650 days, about 660-670 days, about 670-680 days, about 680-690 days, about 690-600 days, about 700-710 days, about 710-720 days, about 720-730 days, about 730-740 days, about 740-750 days, about 760-770 days, about 770-780 days, about 780-790 days, about 790-700 days, about 800-810 days, about 810-820 days, about 820-830 days, about 830-840 days, about 840-850 days, about 860-870 days, about 870-880 days, about 880-890 days, about 890-800 days, about 900-910 days, about 910-920 days, about 920-930 days, about 930-940 days, about 940-950 days, about 960-970 days, about 970-980 days, about 980-990 days, about 990-900 days, or more than about 900 days.

In some embodiments, facultative wintering in Camelina sativa can include, for example, flowering absent vernalization. In some embodiments, the ability of plants to flower absent vernalization is measured. In some embodiments, the ability of plants to flower absent vernalization is measured by, for example, growing plants absent vernalization and recording the day on which each plant produces a first flower. In some embodiments, a plant that produces a first flower within, for example, about 10-20 days, about 20-30 days, about 30-40 days, about 40-50 days, about 50-60 days, about 60-70 days, about 70-80 days, about 80-90 days, about 90-100 days, about 100-110 days, about 110-120 days, about 120-130 days, about 130-140 days, about 140-150 days, about 150-160 days, about 160-170 days, about 170-180 days, about 180-190 days, about 190-200 days, about 200-210 days, about 210-220 days, about 220-230 days, about 230-240 days, about 240-250 days, about 260-270 days, about 270-280 days, about 280-290 days, about 290-300 days, about 300-310 days, about 310-320 days, about 320-330 days, about 330-340 days, about 340-350 days, about 360-370 days, about 370-380 days, about 380-390 days, about 390-300 days, about 400-410 days, about 410-420 days, about 420-430 days, about 430-440 days, about 440-450 days, about 460-470 days, about 470-480 days, about 480-490 days, about 490-400 days, about 500-510 days, about 510-520 days, about 520-530 days, about 530-540 days, about 540-550 days, about 560-570 days, about 570-580 days, about 580-590 days, about 590-500 days, about 600-610 days, about 610-620 days, about 620-630 days, about 630-640 days, about 640-650 days, about 660-670 days, about 670-680 days, about 680-690 days, about 690-600 days, about 700-710 days, about 710-720 days, about 720-730 days, about 730-740 days, about 740-750 days, about 760-770 days, about 770-780 days, about 780-790 days, about 790-700 days, about 800-810 days, about 810-820 days, about 820-830 days, about 830-840 days, about 840-850 days, about 860-870 days, about 870-880 days, about 880-890 days, about 890-800 days, about 900-910 days, about 910-920 days, about 920-930 days, about 930-940 days, about 940-950 days, about 960-970 days, about 970-980 days, about 980-990 days, about 990-900 days of growth absent vernalization is considered able to flower absent vernalization.

In some embodiments, a Camelina sativa plant exhibiting facultative wintering may, for example, have a non-functional allele of the FLOWERING LOCUS C gene on chromosome 20 and flower absent vernalization. In some embodiments, a Camelina sativa plant exhibiting facultative wintering may, for example, have an allele of a flowering time pathway gene that differentiates it from control spring-type Camelina sativa plants. In some embodiments, a Camelina sativa plant exhibiting facultative wintering may, for example, flower and mature later than control spring-type Camelina sativa plants without vernalization in non-freezing environments. In some embodiments, a Camelina sativa plant exhibiting facultative wintering may, for example, survive freezing environments after transitioning to a floral meristem. In some embodiments, a Camelina sativa plant exhibiting facultative wintering may, for example, flower and reach maturity earlier than control winter-type Camelina sativa plants when grown under the same winter conditions in a field in Minnesota.

In some embodiments, a Camelina sativa plant exhibiting facultative wintering may, for example, exhibit resistance to weevils. In some embodiments, the weevils may be of the species Ceutorhynchus erysimi (Shephard's purse weevil). In some embodiments, weevil resistance is increased as compared to that of plants of ‘PI650168’ Camelina sativa grown under the same conditions. In some embodiments, resistance to weevils includes at least about 50%, at least about 60%, at least about 70%, at least about 80%, at least about 90%, or about 90-100% of leaf area not having visible damage after being infected by weevils.

In some embodiments, a Camelina sativa plant exhibiting facultative wintering may, for example, exhibit increased seed area. In some embodiments, increased seed area is measured in mm² and is as compared to the area of seeds from an appropriate control plant (e.g. ‘PI650163-1’ Camelina sativa grown under the same conditions). In some embodiments, a Camelina sativa plant exhibiting facultative wintering may, for example, have seeds with an average seed area in the range of about 0.4 to about 0.6, about 0.6 to about 0.8, about 0.8 to about 1.0, about 1.0 to about 1.2, about 1.2 to about 1.4, about 1.4 to about 1.6, about 1.6 to about 1.8, about 1.8 to about 2.0, about 2.0 to about 2.2, about 2.2 to about 2.4, about 2.4 to about 2.6, about 2.6 to about 2.8, about 2.8 to about 3.0, about 3.0 to about 3.2, about 3.2 to about 3.4, about 3.4 to about 3.6, about 3.8, about 3.8 to about 4.0, or about 4.0 to about 4.2 mm².

In some embodiments, a Camelina sativa plant exhibiting facultative wintering may, for example, exhibit increased seed mass. In some embodiments, increased seed mass is measured in grams per thousand seeds and is as compared to that of seeds from an appropriate control plant (e.g. ‘PI650163-1’ Camelina sativa grown under the same conditions). In some embodiments, a Camelina sativa plant exhibiting facultative wintering may, for example, have seeds with a seed mass in the range of about 0.2 to about 0.3, about 0.3 to about 0.4, about 0.4 to about 0.5, about 0.5 to about 0.6, about 0.6 to about 0.7, about 0.7 to about 0.8, about 0.8 to about 0.9, about 0.9 to about 1.0, about 1.0 to about 1.1, about 1.1 to about 1.2, about 1.2 to about 1.3, about 1.3 to about 1.4, about 1.4 to about 1.5, about 1.5 to about 1.6, about 1.6 to about 1.7, about 1.7 to about 1.8, about 1.8 to about 1.9, about 1.9 to about 2.0, about 2.0 to about 2.1, about 2.1 to about 2.2, about 2.2 to about 2.3, about 2.3 to about 2.4, about 2.4 to about 2.5, about 2.5 to about 2.6, about 2.6 to about 2.7, about 2.7 to about 2.8, about 2.8 to about 2.9, or about 2.9 to about 3.0 grams per thousand seeds.

In some embodiments, a Camelina sativa plant exhibiting facultative wintering may, for example, exhibit increased seed yield. In some embodiments, increased seed yield is measured in kg/ha and is as compared to the seed yield of plants of a control winter-type Camelina sativa line grown under the same conditions. In some embodiments, the control winter-type Camelina sativa line is ‘PI650155’. In some embodiments, a Camelina sativa plant exhibiting facultative wintering may, for example, exhibit a seed yield in the range of about 300 to about 500, about 500 to about 700, about 700 to about 900, about 900 to about 1100, about 1100 to about 1300, about 1300 to about 1500, about 1500 to about 1700, about 1700 to about 1900, about 1900 to about 2100, about 2100 to about 2300, about 2300 to about 2500, about 2500 to about 2700, about 2700 to about 2900, about 2900 to about 3100, about 3100 to about 3300, about 3300 to about 3500, about 3500 to about 3700, about 3700 to about 3900, or about 3900 to about 4100 kg/ha. In some embodiments, a Camelina sativa plant exhibiting facultative wintering may, for example, exhibit a seed yield in the range of about 1100 to about 1300 kh/ha.

In some embodiments, a Camelina sativa plant exhibiting facultative wintering may, for example, exhibit increased plant height. In some embodiments, increased plant height is as compared to plants of ‘PI650163-1’ Camelina sativa when grown under the same conditions. In some embodiments, a Camelina sativa plant exhibiting facultative wintering may, for example, exhibit a plant height in the range of about 15 to about 25, about 25 to about 35, about 35 to about 45, about 45 to about 55, about 55 to about 65, about 65 to about 75, about 75 to about 85, about 85 to about 95, about 95 to about 105, about 105 to about 115, about 115 to about 125, about 125 to about 135, about 135 to about 145, about 145 to about 155, about 155 to about 165, about 165 to about 175, about 175 to about 185, or about 185 to about 195. In some embodiments, a Camelina sativa plant exhibiting facultative wintering may, for example, exhibit a plant height in the range of about 57 cm to about 65 cm.

In some embodiments, a Camelina sativa plant exhibiting facultative wintering may, for example, be derived from a cross between a first Camelina sativa accession and a second Camelina sativa accession. In some embodiments, the first Camelina sativa accession requires vernalization to flower and the second Camelina sativa accession does not require vernalization to flower. In some embodiments, the first Camelina sativa accession is ‘PI650168’ and the second Camelina sativa accession is ‘P1650163-1’. In some embodiments, ‘PI650168’ is the female parent ‘PI650163-1’ is the male parent. In some embodiments, at least one parent has a subfunctional or non-functional allele of FLC on chromosome 20. In some embodiments, a Camelina sativa plant exhibiting facultative wintering may, for example, be present in the F1 progeny of the cross and/or in any subsequent generation produced by selfing.

In some embodiments, a Camelina sativa plant exhibiting facultative wintering may, for example, be produced by a) conducting Kompetitive Allele Specific PCR (KASP) genotyping on a pool of potential parent Camelina sativa accessions, in which primers are chosen for the KASP genotyping that target the SNV in exon 5 of FLC on chromosome 20; b) identifying, from the pool of potential parent Camelina sativa accessions, at least one Camelina sativa accession with a subfunctional or nonfunctional allele of FLC on chromosome 20; c) crossing the Camelina sativa accession with a subfunctional or nonfunctional allele of FLC on chromosome 20 identified in step b) with itself or with another Camelina sativa accession and allowing progeny to be formed; d) measuring, in the progeny of the cross in step c): i) the number of days to reach maturity compared to at least one control winter Camelina sativa accession grown under the same conditions, and ii) the ability to flower absent vernalization; and e) selecting from the progeny a Camelina sativa plant exhibiting the traits of i) early maturity and ii) flowering absent vernalization.

In some embodiments, a Camelina sativa plant exhibiting facultative wintering may, for example, be derived from F1 progeny of a cross between a plant of Camelina sativa accession ‘PI650168’ with a plant of Camelina sativa accession ‘P1650163-1’. In some embodiments, the F1 progeny are optionally allowed to reproduce via selfing for at least one generation to produce additional Camelina sativa plant exhibiting facultative wintering.

A Camelina sativa plant grown from seed deposited under ATCC Accession Number X1 is an exemplary source of the facultative wintering trait disclosed herein. Thus, a sample of seed having been deposited under ATCC Accession Number X1 is representative of a Camelina sativa plant exhibiting the facultative wintering trait disclosed herein. Seed that is “representative of” a Camelina sativa plant exhibiting the facultative wintering trait as disclosed herein need not be genetically identical to the Camelina sativa plant exhibiting the facultative wintering trait so long as the genetic determinants of the facultative wintering trait are consistent between the deposited seeds and the plant exhibiting facultative wintering. For example, a plant need not be directly or indirectly bred from seed deposited under ATCC Accession Number X1 in order for such deposited seed to be considered “representative of” the Camelina sativa plant exhibiting the facultative wintering trait, so long as the Camelina sativa plant exhibits the facultative wintering trait in substantially the same physiological and morphological manners as described herein for Camelina sativa line ‘CMN2207’.

In some embodiments, a Camelina sativa plant exhibiting facultative wintering may, for example, have all the physiological and morphological characteristics of a Camelina sativa plant grown from seed deposited under ATCC Accession Number X1. In some embodiments, a Camelina sativa plant exhibiting facultative wintering may, for example, be derived from a Camelina sativa plant grown from seed deposited under ATCC Accession Number X1.

In aspects of the invention, a Camelina sativa plant exhibiting facultative wintering may, for example, be cultivated in a no-till relay-cropping agricultural system. In some embodiments, the no-till relay-cropping agricultural system has at least two different species of crops planted on different dates. In some embodiments, at least one species is a Camelina sativa and at least one species is Glycine. In some embodiments, the Camelina sativa plant exhibit facultative wintering. In some embodiments, the Camelina sativa crop is planted before the Glycine crop. In some embodiments, the plants of the Glycine crop are less damaged on average during harvest of the facultative wintering Camelina sativa crop than are Glycine plants grown in a control no-till relay-cropping agricultural system with control winter-type Camelina sativa plants and the same Glycine plants.

Methods of Making Camelina sativa Plants Having the Facultative Wintering Trait and Seeds of the Same

In some aspects, the present disclosure relates to a method of making a Camelina sativa plant having the facultative wintering trait, said method including crossing or self-pollinating the Camelina sativa plant having the facultative wintering trait of any one of the preceding embodiments that have a Camelina sativa plant having the facultative wintering trait with another Camelina sativa plant having the facultative wintering trait and harvesting seed therefrom. All plants produced using the Camelina sativa plant having the facultative wintering trait as at least one parent are within the scope of this disclosure, including those developed from cultivars derived from the Camelina sativa plant having the facultative wintering trait. Advantageously, the Camelina sativa plant having the facultative wintering trait cultivar could be used in crosses with other, different, Camelina sativa plants to produce the first generation (F₁) Camelina sativa hybrid seeds and plants with superior characteristics. The Camelina sativa plant having the facultative wintering trait of the disclosure can also be used for transformation where exogenous genes are introduced and expressed by the Camelina sativa plant having the facultative wintering trait of the disclosure. Genetic variants created either through traditional breeding methods using the Camelina sativa plant having the facultative wintering trait or through transformation of the Camelina sativa plant having the facultative wintering trait by any of a number of protocols known to those of skill in the art are intended to be within the scope of this disclosure.

The following describes breeding methods that may be used with the Camelina sativa plant having the facultative wintering trait in the development of further Camelina sativa plants. One such embodiment is a method for developing progeny Camelina sativa plants in a Camelina sativa plant breeding program including: obtaining the Camelina sativa plant, or a part thereof, of the Camelina sativa plant having the facultative wintering trait, utilizing said plant or plant part as a source of breeding material, and selecting a progeny plant with molecular markers in common with the Camelina sativa plant having the facultative wintering trait and/or with morphological and/or physiological characteristics selected from the characteristics listed above. Breeding steps that may be used in the Camelina sativa plant breeding program include pedigree breeding, backcrossing, mutation breeding, and recurrent selection. In conjunction with these steps, techniques such as RFLP-enhanced selection, genetic marker enhanced selection (for example, SSR markers) and the making of double haploids may be utilized.

Another method involves producing a population of progeny Camelina sativa plants, by crossing the Camelina sativa plant having the facultative wintering trait with another Camelina sativa plant, thereby producing a population of Camelina sativa plants, which, on average, derive 50% of their alleles from the Camelina sativa plant having the facultative wintering trait. A plant of this population may be selected and repeatedly selfed or sibbed with a Camelina sativa plant resulting from these successive filial generations. One embodiment of this disclosure is the Camelina sativa cultivar produced by this method and that has obtained at least 50% of its alleles from the Camelina sativa plant having the facultative wintering trait.

Additional methods include, but are not limited to, expression vectors introduced into plant tissues using a direct gene transfer method, such as microprojectile-mediated delivery, DNA injection, electroporation, and the like. More preferably, expression vectors are introduced into plant tissues by using either microprojectile-mediated delivery with a biolistic device or by using Agrobacterium-mediated transformation. Transformed plants obtained with the protoplasm of the disclosure are intended to be within the scope of this disclosure.

Additional methods include, without limitation, chasing selfs. Chasing selfs involves identifying inbred plants among Camelina sativa plants that have been grown from hybrid Camelina sativa seed. Once the seed is planted, the inbred plants may be identified and selected due to their decreased vigor relative to the hybrid plants that grow from the hybrid seed. By locating the inbred plants, isolating them from the rest of the plants, and self-pollinating them (i.e., “chasing selfs”), a breeder can obtain an inbred line that is identical to an inbred parent used to produce the hybrid.

Accordingly, another aspect of the present disclosure relates a method for producing an inbred Camelina sativa variety by: planting seed of the Camelina sativa plant having the facultative wintering trait; growing plants from the seed; identifying one or more inbred Camelina sativa plants; controlling pollination in a manner which preserves homozygosity of the one or more inbred plants; and harvesting resultant seed from the one or more inbred plants. The step of identifying the one or more inbred Camelina sativa plants may further include identifying plants with decreased vigor, i.e., plants that appear less robust than plants of the Camelina sativa plant having the facultative wintering trait. Camelina sativa plants capable of expressing substantially all of the physiological and morphological characteristics of the parental inbred lines of the Camelina sativa plant having the facultative wintering trait include Camelina sativa plants obtained by chasing selfs from seed of the Camelina sativa plant having the facultative wintering trait.

One of ordinary skill in the art will recognize that once a breeder has obtained inbred Camelina sativa plants by chasing selfs from seed of the Camelina sativa plant having the facultative wintering trait, the breeder can then produce new inbred plants such as by sib-pollinating, or by crossing one of the identified inbred Camelina sativa plant with a plant of the Camelina sativa plant having the facultative wintering trait.

One of ordinary skill in the art of plant breeding would know how to evaluate the traits of two plant varieties to determine if there is no significant difference between the two traits expressed by those varieties. For example, see Fehr and Walt, Principles of Cultivar Development, pp. 261-286 (1987). Thus, the disclosure includes a progeny plant of the Camelina sativa plants of any of the above embodiments including a combination of at least two of the Camelina sativa plants selected from the group of those listed above, so that said progeny Camelina sativa plant is not significantly different for said traits than the Camelina sativa plant having the facultative wintering trait as determined at the 5% significance level when grown in the same environmental conditions. Using techniques described herein, molecular markers may be used to identify said progeny plant as a Camelina sativa plant having the facultative wintering trait progeny plant. Mean trait values may be used to determine whether trait differences are significant, and preferably the traits are measured on plants grown under the same environmental conditions. Once such a variety is developed its value is substantial since it is important to advance the germplasm base as a whole in order to maintain or improve traits such as yield, disease resistance, pest resistance, and plant performance in extreme environmental conditions.

Progeny of the Camelina sativa plant having the facultative wintering trait may also be characterized through their filial relationship with the Camelina sativa plant having the facultative wintering trait, as for example, being within a certain number of breeding crosses of the Camelina sativa plant having the facultative wintering trait. A breeding cross is a cross made to introduce new genetics into the progeny, and is distinguished from a cross, such as a self or a sib cross, made to select among existing genetic alleles. The lower the number of breeding crosses in the pedigree, the closer the relationship between the Camelina sativa plant having the facultative wintering trait and its progeny. For example, progeny produced by the methods described herein may be within 1, 2, 3, 4, or 5 breeding crosses of the Camelina sativa plant having the facultative wintering trait.

### Plant Breeding and Product Development

Camelina sativa is an important and valuable oilseed plant. Thus, a continuing goal of Camelina sativa plant breeders is to develop stable, attractive hybrid Camelina sativa that are agronomically sound. To accomplish this goal, the Camelina sativa breeder must select and develop Camelina sativa plants with traits that result in superior cultivars.

Proper testing should detect any major faults and establish the level of superiority or improvement over current cultivars. In addition to showing superior performance, there must be a demand for a new cultivar that is compatible with industry standards or which creates a new market. The introduction of a new cultivar will incur additional costs to the seed producer, the grower, processor and consumer for special advertising and marketing, altered seed and commercial production practices, and new product utilization. The testing preceding release of a new cultivar should take into consideration research and development costs, as well as technical superiority of the final cultivar. For seed-propagated cultivars, it must be feasible to produce seed easily and economically.

Choice of breeding or selection methods depends on the mode of plant reproduction, the heritability of the trait(s) being improved, and the type of cultivar used commercially (e.g., Fi hybrid cultivar, pure line cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, the overall value of the advanced breeding lines, and the number of successful cultivars produced per unit of input (e.g., per year, per dollar expended, etc.).

Promising advanced breeding lines are thoroughly tested and compared to appropriate standards in environments representative of the commercial target area(s) for at least three years. The best lines are candidates for new commercial cultivars. Those still deficient in a few traits are used as parents to produce new populations for further selection.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

The goal of Camelina sativa plant breeding is to develop new, unique, and superior hybrid Camelina sativa accessions. The breeder initially selects and crosses two or more parental lines, followed by repeated selfing and selection, producing many new genetic combinations. The breeder can theoretically generate billions of different genetic combinations via crossing, selfing, and mutations. The breeder has no direct control at the cellular level. Therefore, two breeders will never develop the same line, or even very similar lines, having the same Camelina sativa traits.

Each year, the plant breeder selects the germplasm to advance to the next generation. This germplasm is grown under different geographical, climatic, and soil conditions, and further selections are then made during, and at the end of, the growing season. The cultivars that are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce the same line twice by using the exact same original parents and the same selection techniques. This unpredictability results in the expenditure of large research monies to develop superior hybrid Camelina sativa accessions.

The development of commercial Camelina sativa accessions requires the development of Camelina sativa varieties, the crossing of these varieties, and the evaluation of the crosses. Pedigree breeding and recurrent selection breeding methods are used to develop cultivars from breeding populations. Breeding programs combine desirable traits from two or more varieties or various broad-based sources into breeding pools from which cultivars are developed by selfing and selection of desired phenotypes. The new cultivars are crossed with other varieties and the hybrids from these crosses are evaluated to determine which have commercial potential.

Pedigree breeding is used commonly for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population. Then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines with each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs-which are also referred to as Microsatellites), and Single Nucleotide Polymorphisms (SNPs).

Isozyme Electrophoresis and RFLPs have been widely used to determine genetic composition. Shoemaker and Olsen (Molecular Linkage Map of Soybean (Glycine max), pp. 6.131-6.138 in S. J. O'Brien (ed.) Genetic Maps: Locus Maps of Complex Genomes, Cold Spring Harbor Laboratory Press, Cold Spring Harbor, N.Y. (1993)) developed a molecular genetic linkage map that consisted of 25 linkage groups with about 365 RFLP, 11 RAPD, three classical markers, and four isozyme loci. See also, Shoemaker, R. C., RFLP Map of Soybean, pp. 299-309, in Phillips, R. L. and Vasil, I. K. (eds.), DNA-Based Markers in Plants, Kluwer Academic Press, Dordrecht, the Netherlands (1994).

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus in soybean with as many as 26 alleles. Diwan, N. and Cregan, P. B., Theor. Appl. Genet., 95:22-225 (1997). SNPs may also be used to identify the unique genetic composition of the disclosure and progeny varieties retaining that unique genetic composition. Various molecular marker techniques may be used in combination to enhance overall resolution.

Molecular markers, which include markers identified through the use of techniques such as Isozyme Electrophoresis, RFLPs, RAPDs, AP-PCR, DAF, SCARs, AFLPs, SSRs, and SNPs, may be used in plant breeding. One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding is another method of introducing new traits into Camelina sativa varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid, or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company (1993).

The production of double haploids can also be used for the development of homozygous varieties in a breeding program. Double haploids are produced by the doubling of a set of chromosomes from a heterozygous plant to produce a completely homozygous individual. For example, see Wan, et al., Theor. Appl. Genet., 77:889-892 (1989).

Descriptions of other breeding methods that are commonly used for different traits and crops can be found in one of several reference books (e.g., Principles of Plant Breeding, John Wiley and Son, pp. 115-161 (1960); Allard (1960); Simmonds (1979); Sneep, et al. (1979); Fehr (1987); “Carrots and Related Vegetable Umbelliferae,” Rubatzky, V. E., et al. (1999).

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Any DNA sequences, whether from a different species or from the same species, which are introduced into the genome using transformation or various breeding methods, are referred to herein collectively as “transgenes.” Over the last fifteen to twenty years, several methods for producing transgenic plants have been developed, and the present disclosure, in particular embodiments, also relates to transformed versions of the claimed line.

Nucleic acids or polynucleotides refer to RNA or DNA that is linear or branched, single or double stranded, or a hybrid thereof. The term also encompasses RNA/DNA hybrids. These terms also encompass untranslated sequence located at both the 3′ and 5′ ends of the coding region of the gene: at least about 1000 nucleotides of sequence upstream from the 5′ end of the coding region and at least about 200 nucleotides of sequence downstream from the 3′ end of the coding region of the gene. Less common bases, such as inosine, 5-methylcytosine, 6-methyladenine, hypoxanthine, and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The antisense polynucleotides and ribozymes can consist entirely of ribonucleotides, or can contain mixed ribonucleotides and deoxyribonucleotides. The polynucleotides of the disclosure may be produced by any means, including genomic preparations, cDNA preparations, in vitro synthesis, RT-PCR, and in vitro or in vivo transcription.

Plant transformation involves the construction of an expression vector that will function in plant cells. Such a vector includes DNA including a gene under control of, or operatively linked to, a regulatory element (for example, a promoter). The expression vector may contain one or more such operably linked gene/regulatory element combinations. The vector(s) may be in the form of a plasmid, and can be used alone or in combination with other plasmids, to provide transformed Camelina sativa plants using transformation methods known in the art to incorporate transgenes into the genetic material of the Camelina sativa plant(s).

### Overview of the Variety ‘CMN2207’

Certain aspects of the present disclosure relate to Camelina sativa variety ‘CMN2207’. Camelina sativa variety ‘CMN2207’ is an exemplary variety that exhibits the facultative wintering trait.

Camelina sativa variety ‘CMN2207’ was derived from a cross between the female parent ‘PI650168’ (unpatented) and the male parent ‘P1650163-1’ (unpatented) in St. Paul, Minnesota, USA and adapted to the south of Morris, Minnesota, USA and in American Falls, Idaho, USA.

Camelina sativa variety ‘CMN2207’ has shown uniformity and stability for the traits, within the limits of environmental influence for the traits. It has been increased with continued observation for uniformity. No variant traits have been observed or are expected in variety ‘CMN2207’.

### Objective Description of the Variety ‘CMN2207’

The data which define these characteristics is based on observations taken in trials located in St. Paul, Minnesota, USA, at approximately 45 degrees latitude and −93 degrees longitude.
Camelina variety ‘CMN2207’ has the following morphologic and other characteristics:

### Plant:

Plant height at full flowering: Short to medium

### Leaves:

Green color of leaf: Medium
Hairiness on upper part: Present
Dentation of Margin: Absent or very weak

### Timing:

Time of flowering (50% of plants with one opened flower at least) in agronomic setting with vernalization: Very early to early; 226 days from planting
Time of flowering (50% of plants with one opened flower at least) when planted in a growth chamber without vernalization: Medium
Time of fruit ripening in agronomic setting with vernalization: Early; 276 days from planting
Time of fruit ripening when planted in a growth chamber without vernalization: Medium to late
When planted in an agronomic setting in September or October in the Northern USA, Camelina sativa variety ‘CMN2207’ survives winter and flowers and matures earlier than any other winter growth habit line of camelina within UMN germplasm collection. When planted in a growth chamber without vernalization, Camelina sativa variety ‘CMN2207’ first flowers at approximately 43 days after planting, which is relatively late compared to camelina lines with a spring growth habit, which typically flower 30-35 days after planting according to Hotton et al. (Hotton et al. (2020), Phenotypic examination of Camelina sativa (L.) Crantz accessions from the USDA-ARS national genetics resource program, Plants, 9(5), 642).

### Stem:

Total length (including infructescence): Short to medium; 58.1 cm
Diameter (at midpoint): Medium to wide; 4.03 mm
Length (excluding infructescence): Short to medium; 30.8 cm
Number of nodes: Medium; 16.67 nodes

### Silicula:

Length: Short to medium; 0.97 cm
Length of Peduncle: Medium; 1.7 cm

### Seed:

Seed weight: 0.88 g/1000 seeds
Compared to lines of camelina with a spring growth habit, the grams per 1,000 seed weight of Camelina sativa variety ‘CMN2207’ is relatively less (low to medium), but compared to lines of camelina with a winter growth habit, Camelina sativa variety ‘CMN2207’ has a high (medium to high) grams per 1,000 seed weight.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

### Example 1: Development of ‘CMN2207’

The following example describes an exemplary breeding method used to develop facultative wintering Camelina sativa line ‘CMN2207’.

### Materials and Methods

On May 30, 2019, a controlled pollination was made between winter camelina accession PI650168 (female) and facultative camelina accession PI650163-1 (male). On Feb. 4, 2020, 62 F₂ progeny from this cross were planted into 5.08 cm diameter×17.8 cm length Deepots™ and placed in two 61×30.5×17.1 cm pot racks in the greenhouse. The plants were moved to a 4° C. cold room on Feb. 27, 2020, and were placed back into the greenhouse on Mar. 19, 2020. Due to Covid-19 challenges, on Mar. 23, 2020, the plants were moved to a private residence and placed on a south-facing porch with white fluorescent lighting (AgroMax® F54T5HO 6400 k fluorescent lamps) and 16 h/8 h days/nights. The date on which a plant first flowered was recorded until May 14, 2020—less than half of the plants had not flowered by this date and were considered late flowering, while most plants succumbed to a pathogen that began to emerge on Apr. 26, 2020. Eight F₂ plants produced F₃ seed, and the plant that flowered the earliest was selected for a seed increase and 49 F₃ seeds were planted in a zipseal bag on Jun. 26, 2020, which was placed in a growth chamber with white fluorescent lighting (AgroMax® F54T5HO 6400 k fluorescent lamps) and 16 h/8 h days/nights. On Jun. 30, 2020, the zipseal bag was placed in a 4° C. cold room. On Jul. 22, 2020, the 49 vernalized F₃ seedlings were transplanted into 5.08 cm diameter×17.8 cm length Deepots™ and placed in a 61×30.5×17.1 cm pot rack in a growth chamber with white fluorescent lighting (AgroMax® F54T5HO 6400 k fluorescent lamps) and 16 h/8 h days/nights. The 49 F₃ plants were harvested for F₄ seed and head rows were planted in the field in St. Paul on Sept. 23, 2020 using a Planet Jr. cone-seeder with approximately 100 seeds (used a custom-made scoop) per 1.2 meter head row. Twenty individual plants were harvested from each of the 49 head rows, and then the rest of each head row was harvested in bulk. This head row bulked F₄:F₅ seed for 9 of the 49 lines (named CMN2201-CMN2209) was planted in a yield trial in St. Paul, MN on Sep. 15, 2021. Line CMN2207 was selected to also be planted in September 2021 in Morris, Minnesota, USA, and in collaboration with a camelina seed company, in American Falls, Idaho, USA.

### Results and Discussion

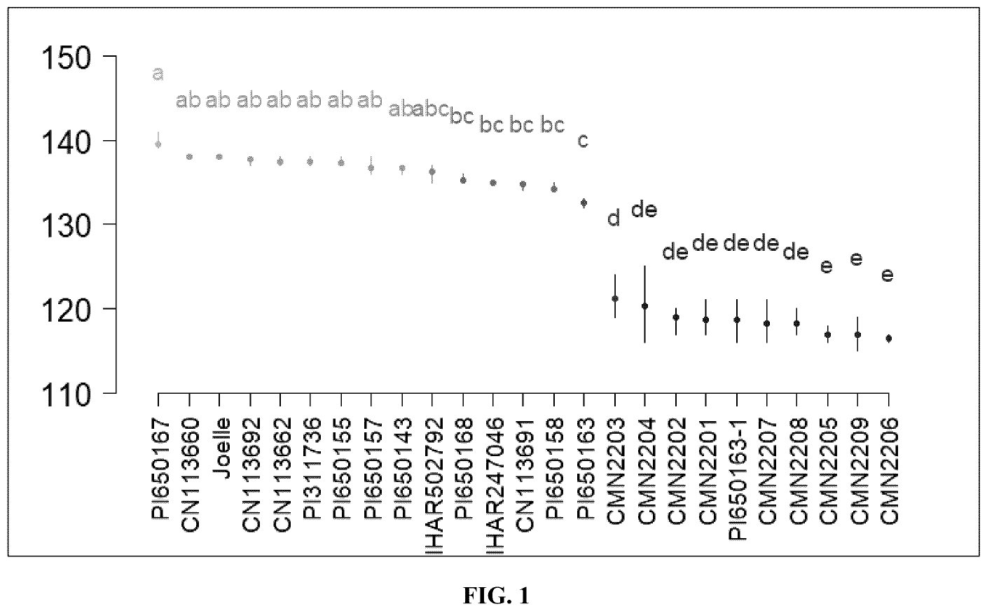

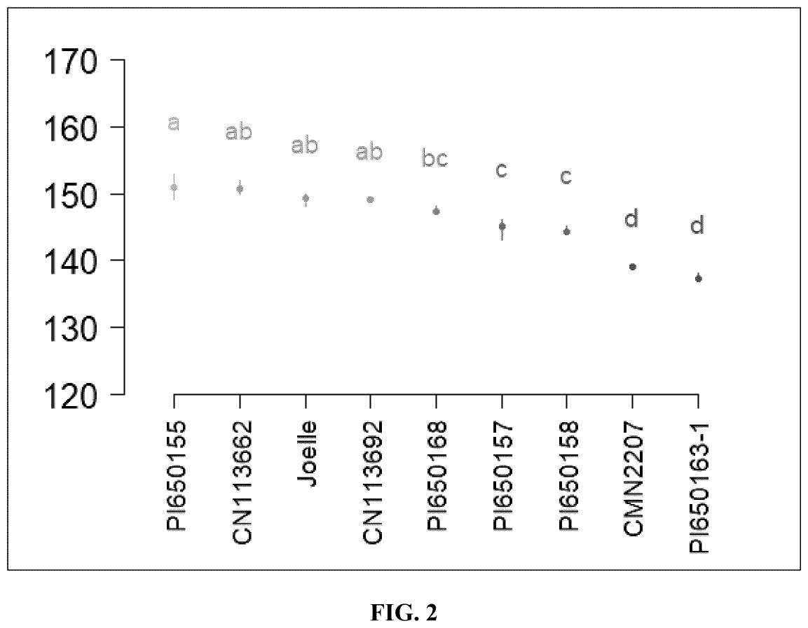

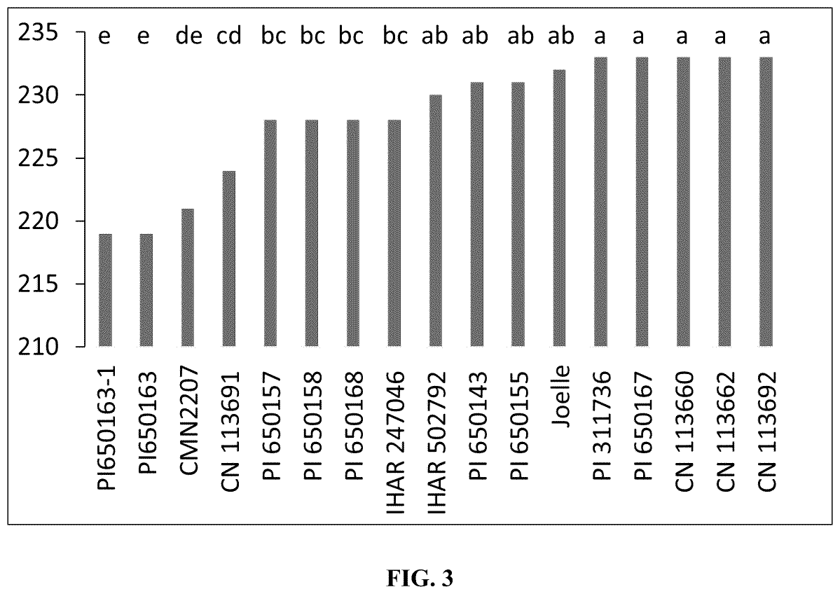

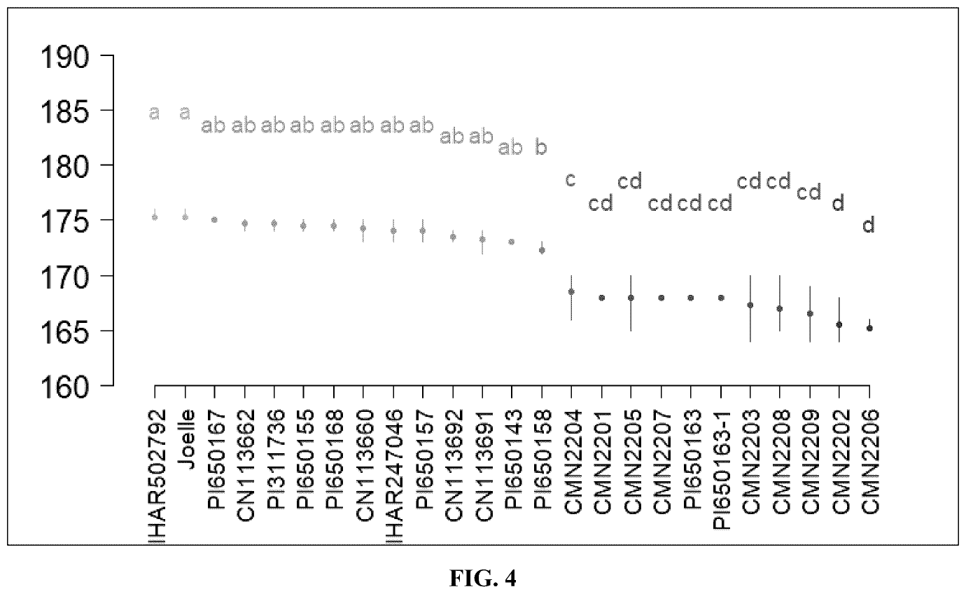

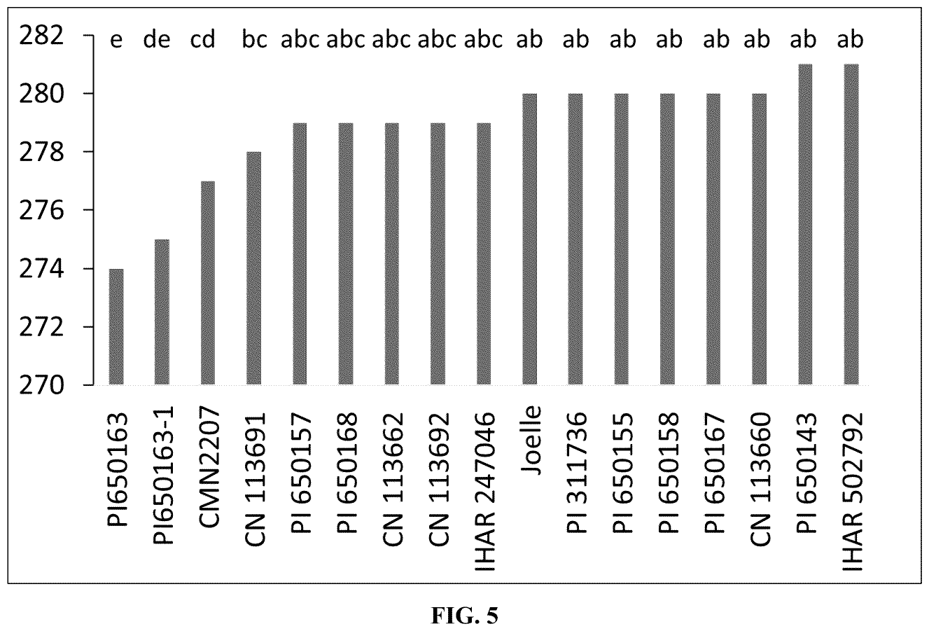

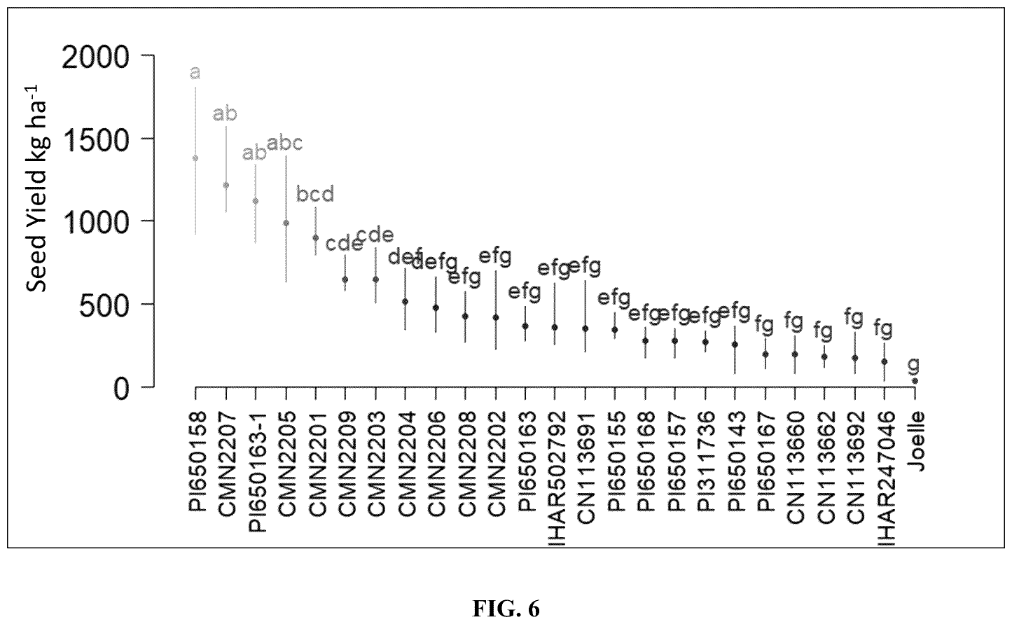

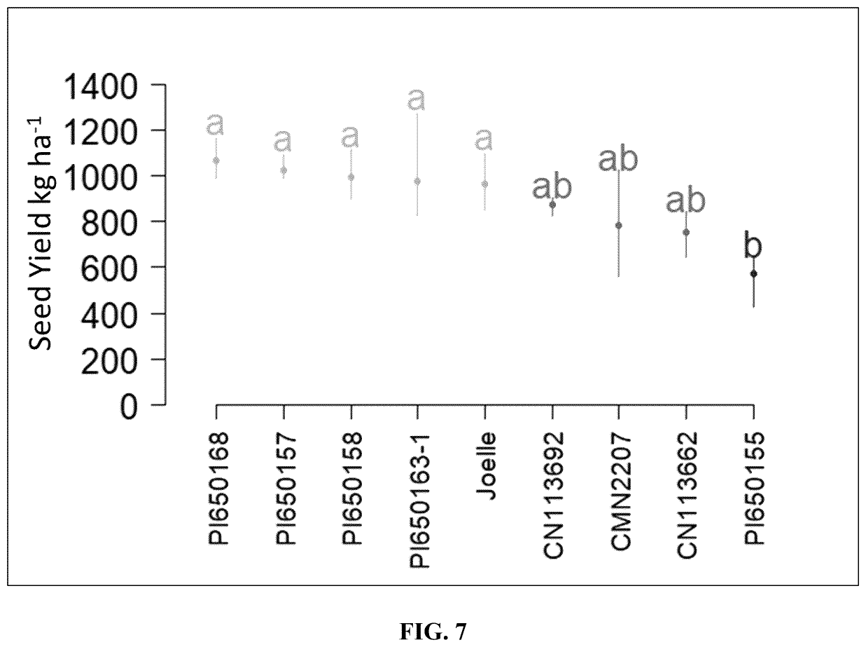

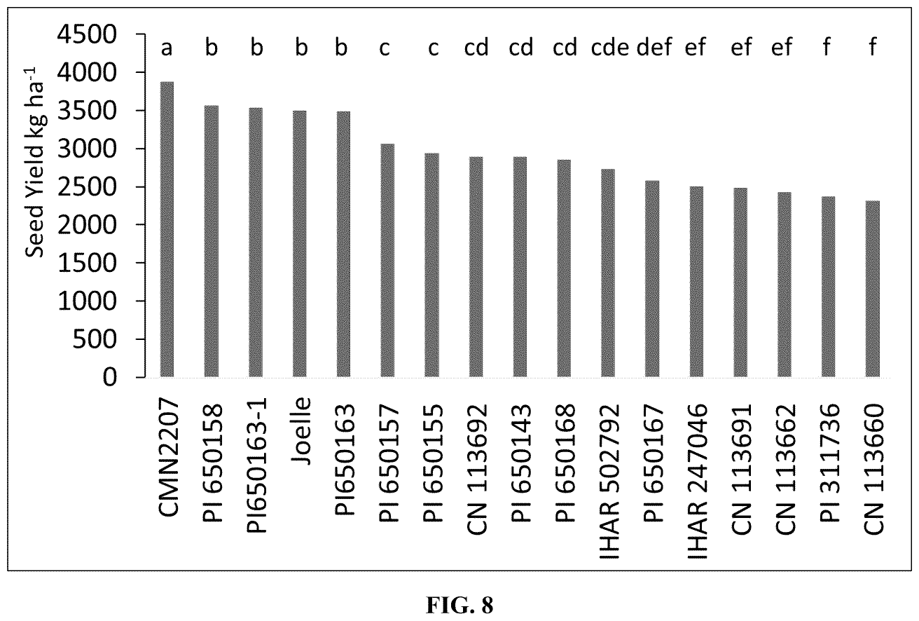

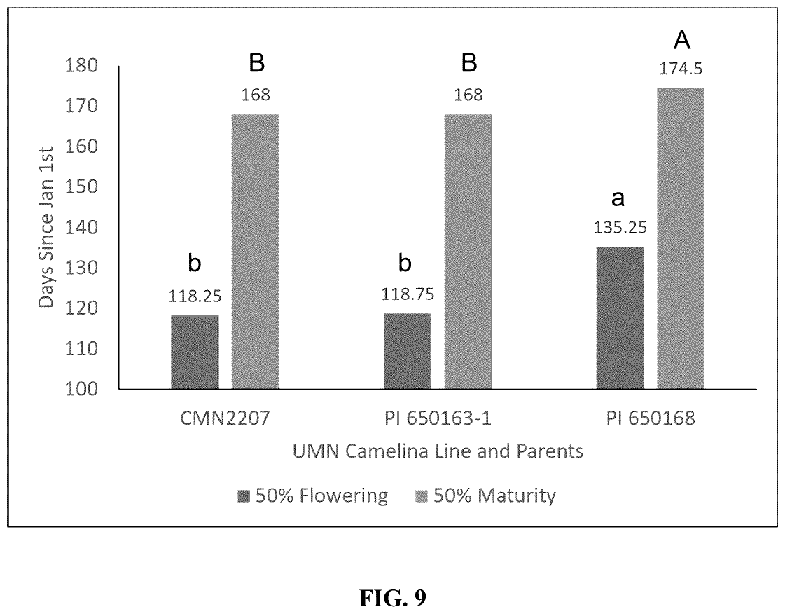

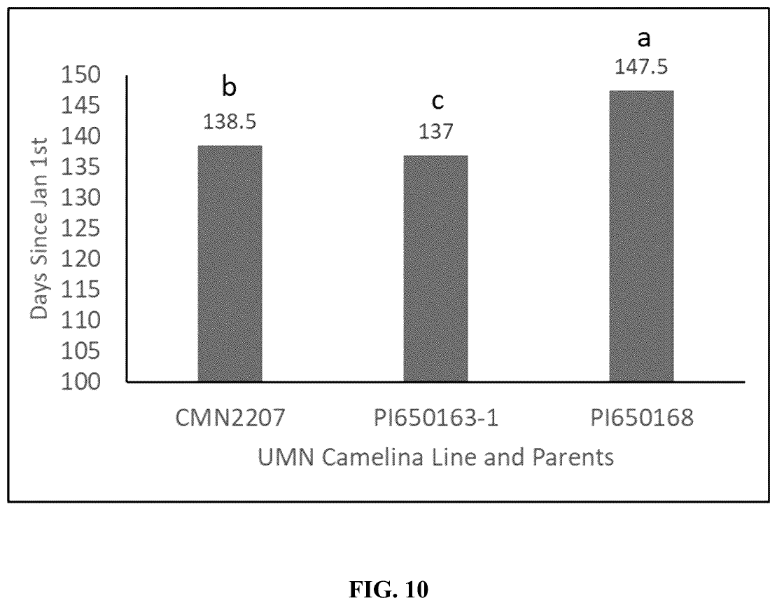

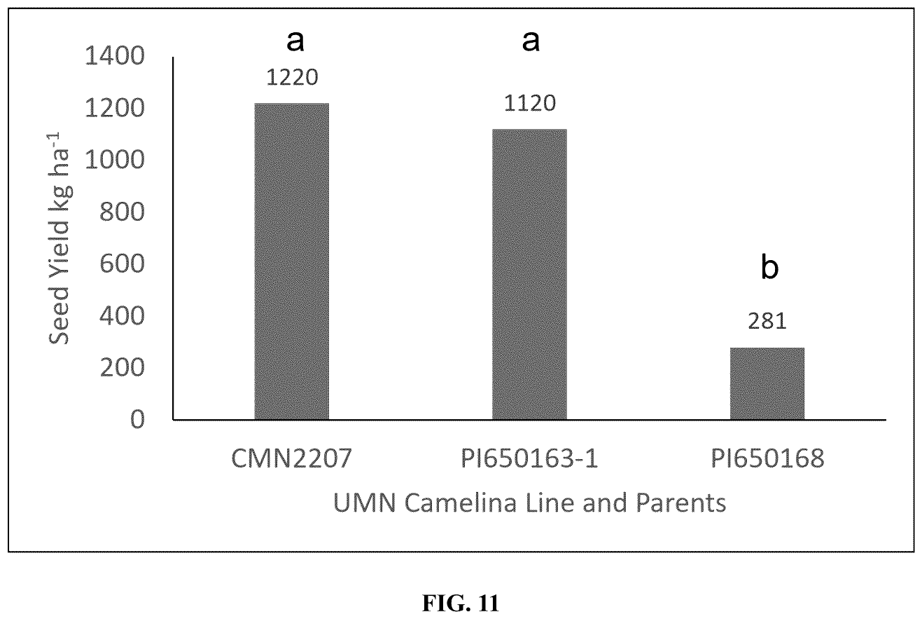

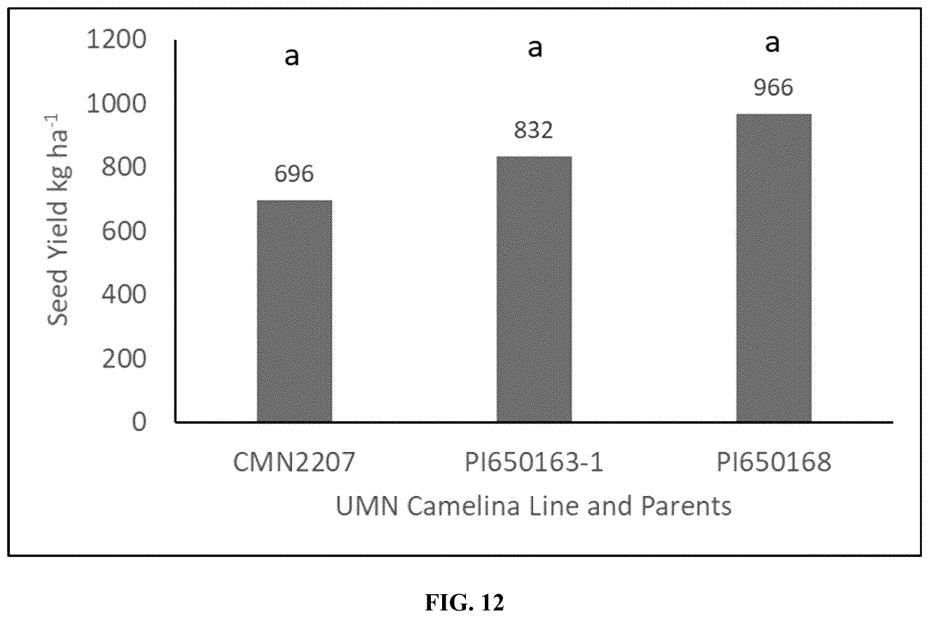

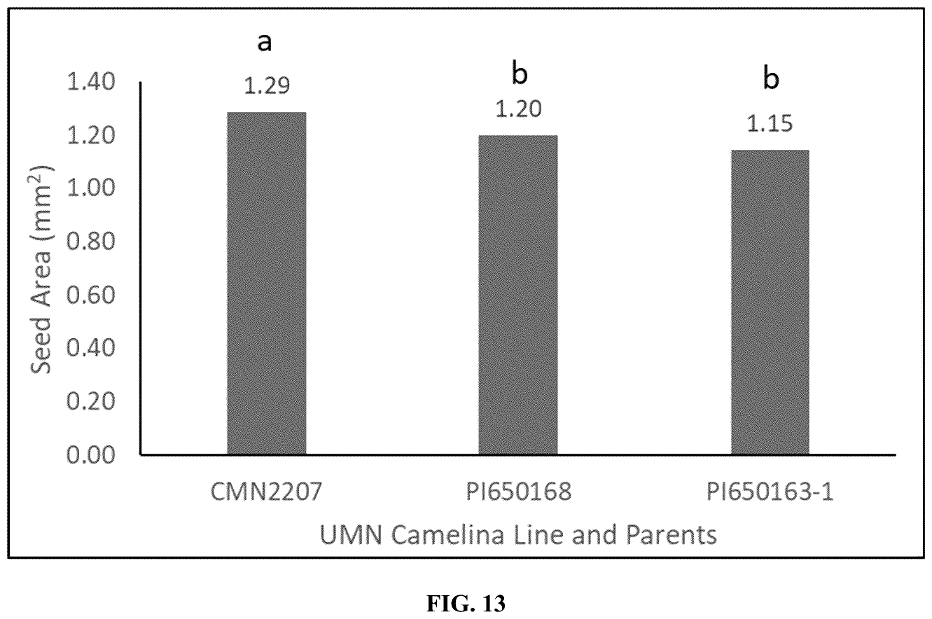

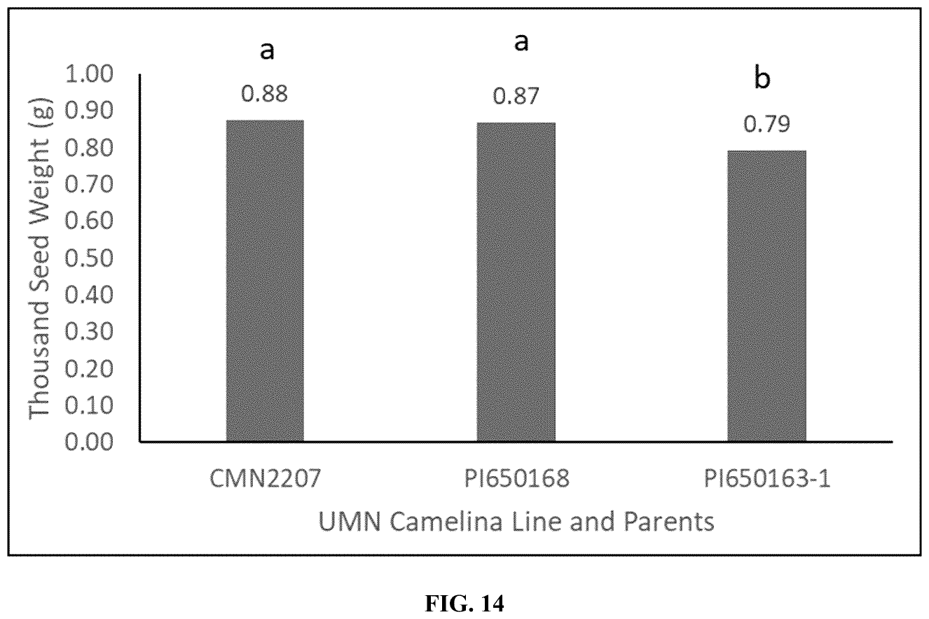

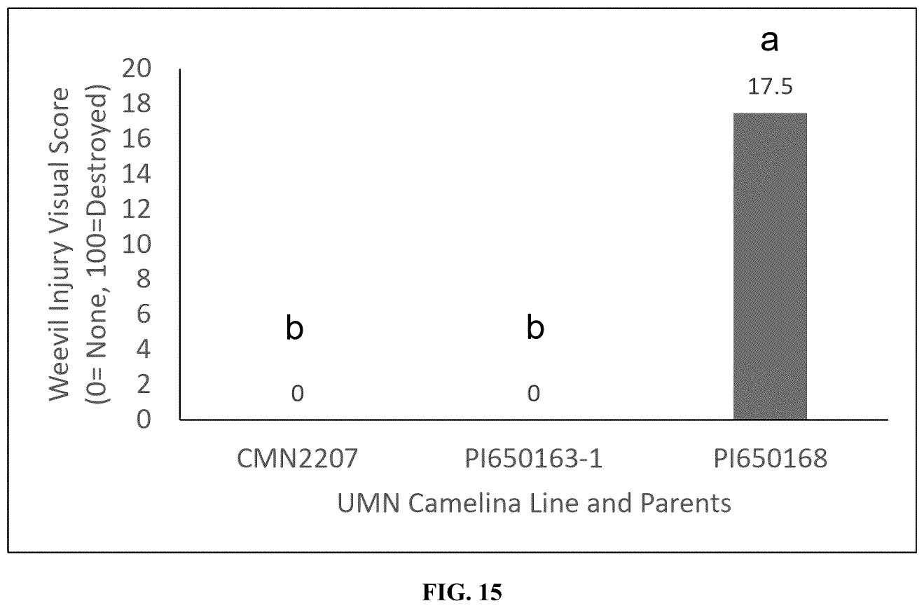

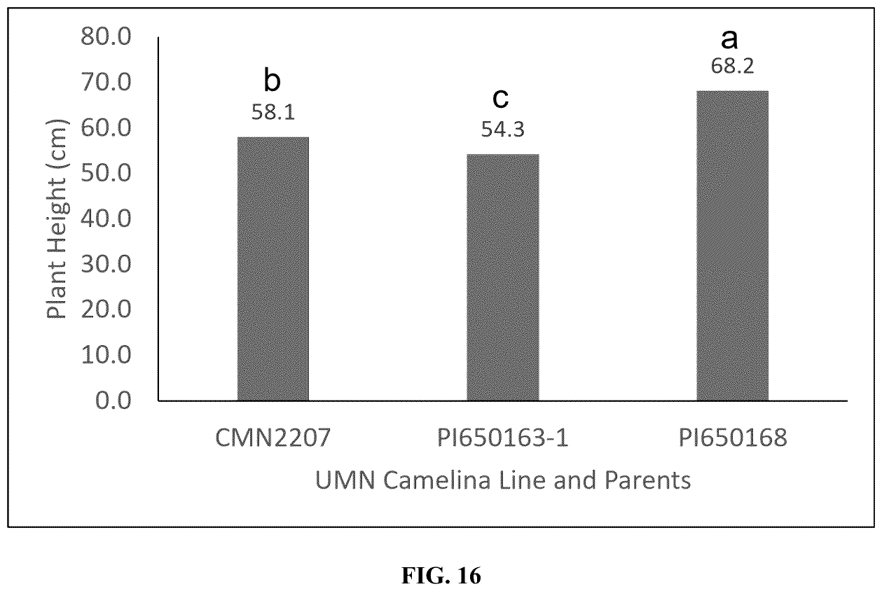

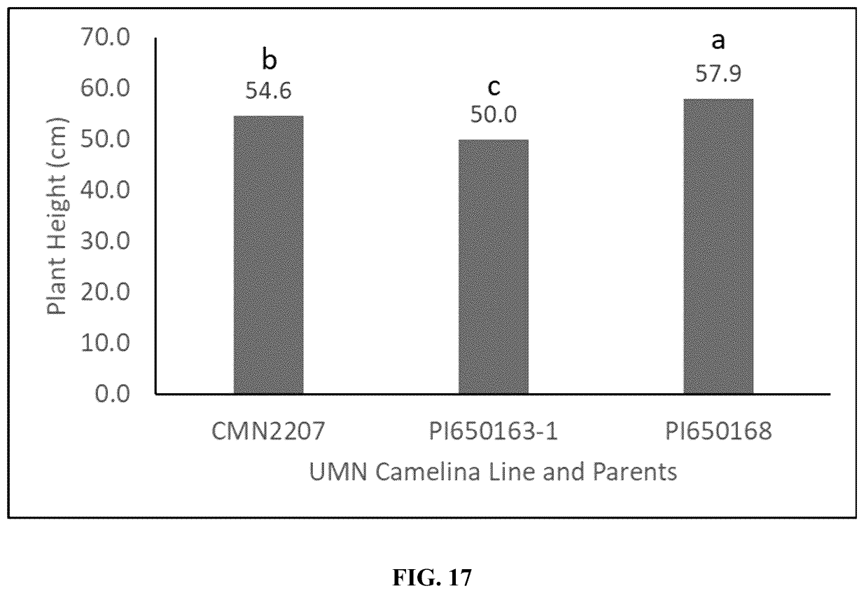

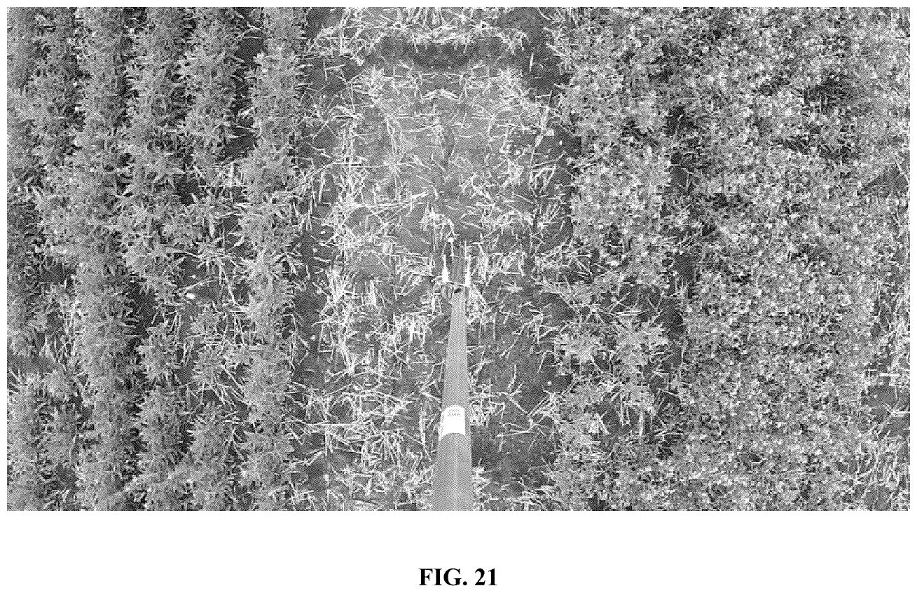

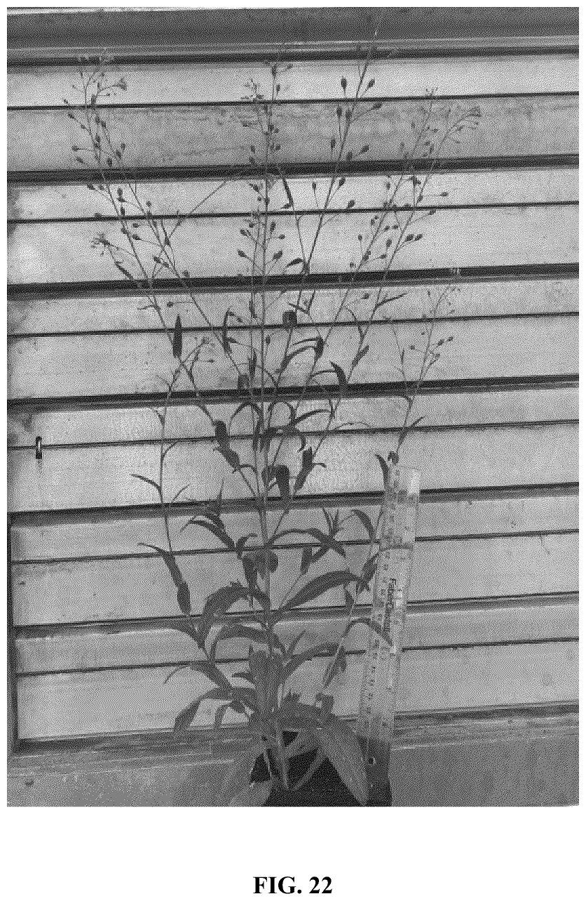

Camelina line CMN2207 flowered earlier than all other winter lines of camelina and was on par with its facultative parent PI650163-1 in St. Paul, Minnesota, USA; Morris, Minnesota, USA; and American Falls, Idaho, USA, in 2022 (FIG. 1, FIG. 2, FIG. 3, FIG. 21). Likewise, it matured earlier than all other winter lines of camelina and was on par with its facultative parent where observations were made in St. Paul and American Falls, Idaho, USA in 2022 (FIG. 4, FIG. 5). CMN2207 had the greatest seed yield among its sibling lines and ranked second overall in St. Paul in 2022 (FIG. 6); while in Morris it yielded moderately well (FIG. 7), and had the greatest seed yield of all other lines in American Falls, ID (FIG. 8). A more detailed comparison among CMN2207 and its parents PI650163-1 (facultative growth habit) and PI650168 (winter growth habit) that includes additional traits such as plant stature and seed size in provided in FIGS. 9-17. A photograph of a CMN2207 plant at flowering stage is provided in FIG. 22. To summarize of these comparisons, CMN2207 is an improvement over its parent PI650168 in terms of flowering, maturity, and weevil resistance, and an improvement over its parent PI650163-1 in terms of seed area, seed mass, and plant height. Larger seeds are preferred because they are less likely to be lost during combine harvest. Taller plants are preferred in a relay-cropping system to ensure separation between camelina and soybean during harvest.

### Example 2: Facultative Winter Accession of Camelina sativa (L. Crantz) with Early Maturity Contributes to Understanding of the Role of FLOWERING LOCUS C in Camelina Flowering

The following example describes how a facultative winter accession of Camelina sativa (L. Crantz) with early maturity contributes to understanding of the role of FLOWERING LOCUS C in camelina flowering.

Camelina is being developed as a winter oilseed cover crop. Early flowering and maturity are desired traits in camelina to allow for relay planting or seeding of a summer annual following camelina harvest. Here it is reported that while all winter biotype accessions of camelina have a functional allele of FLOWERING LOCUS C (FLC) on chromosome 20, there are also at least 20 previously characterized spring biotype accessions that have a functional FLC allele at this locus. This was observed through analysis of 75 accessions (67 spring type, one facultative, and seven winter type) that were resequenced by Li et al., (2020) as well as 11 additional accessions (four spring type, two facultative, and five winter type) that were resequenced for this analysis. Furthermore, a KASP genotyping approach was optimized that effectively differentiates the presence of either the functional or subfunctional FLC allele on chromosome 20. These analyses identified a facultative winter biotype accession of camelina (PI650163-1, winter hardy with subfunctional chromosome 20 FLC allele) that has demonstrated two years of winter-hardiness and has flowered at least a week earlier than the common winter accession, ‘Joelle’. Without wishing to be bound by theory, early maturing winter-hardy camelina is expected to reduce stress on a subsequent soybean crop and improve total cropping system yields when camelina and soybean are grown sequentially in the same season on the same land.

### INTRODUCTION

There is unprecedented global demand for biofuels and feed byproducts as consumers seek out fossil fuel alternatives to mitigate climate change and energy price volatility. The energy producer Exxon Mobil has partnered with Global Clean Energy to purchase 220 million gallons of oil from spring camelina in the western US (Sustainable Oils Camelina (susoils.com) 2022). In the Upper Midwest, winter camelina is being developed as a new cover/cash crop that can be planted in the fall and harvested in spring on the same lands used for traditional summer crop such as that undergoing the maize to soybean rotation on much the Midwestern farm acreage. Without wishing to be bound by theory, it is expected that when planted as a winter annual oilseed cover crop, Camelina sativa (L. Crantz) should be able to ease some of the supply shortage issues for both the energy and feed sectors. Fall planted/spring harvested camelina as an oilseed cover crop also provides important ecosystem services, such as reducing nitrate leaching and providing supplemental nutrition for pollinators, further promoting the crop as a potential solution to several complex challenges (Weyers et al., 2019; Eberle et al., 2015).

Camelina sativa has been shown to be descended from Camelina macrocarpa, with a region of origin spanning the Mediterranean and Eastern Europe (Brock et al., 2018; Chaudhary et al., 2020). It remains unclear exactly when Camelina sativa began to be actively cultivated rather than being tolerated as a weed in cultivated flax (Brock et al., 2018), but its use as a crop can be documented as far back as the early Iron Age (˜1200 BCE) (Zohary et al., 2012). Two cytotypes exist for allohexaploid Camelina sativa—either n=6+7+7 (20) or n=6+6+7 (19) (Hotton et al., 2020). The genome size was estimated to be 785 Mb, which is relatively small for a hexaploid species (Kagale et al., 2014). Camelina is closely related to Arabidopsis thaliana as both are in Brassicacea lineage group I (Huang et al., 2016). Similar to Arabidopsis, camelina is largely a self-fertilized species. In the Camelina sativa germplasm within the US's National Genetic Resources Program, only eight of the 41 accessions exhibited the winter biotype, while the rest exhibited the spring biotype and are not winter-hardy (Hotton et al., 2020). This leaves plant breeders focused on winter types for their value as a winter cover crop with a narrow genetic base. To increase genetic diversity for key traits in winter camelina, one strategy is to cross-pollinate winter and spring types, and either select for winter types in the field if feasible, or backcross as necessary to recover the winter growth habit while introducing impactful alleles. Identifying genetic markers that can predict the winter/spring growth habits in Camelina sativa would save time otherwise spent waiting for populations to phenotypically segregate for this trait before selection and desirable allele stacking could take place in a breeding program. In Arabidopsis, a functional Flowering Locus C (FLC) gene was found to impart a requirement for vernalization ahead of flowering, which the hallmark of a winter type. Similarly, in camelina, the presence of functional or subfunctional FLC orthologs have been shown to effectively differentiate between two spring accessions of camelina (DH55 and CO46) and one winter accession (Ames33292, ‘Joelle’) using qRT-PCR (Anderson et al., 2018). These analyses identified a SNP in FLC on chromosome 20 that distinguishes between the spring and winter habits. This information was used in the present disclosure to optimize a genotyping approach that differentiates winter from spring camelina accessions based upon the chromosome 20 FLC SNP using DNA-based Kompetitive Allele Specific PCR (KASP). To expand the information on the importance of this SNP in controlling flowering time, 11 camelina accessions were resequenced, and an additional 75 resequenced accessions from data on NCBI provided by Li et al., (2020) were downloaded. While this FLC SNP helped differentiate between most spring and winter lines, several spring lines with the winter type FLC allele were identified.

### Materials and Methods

Field and Indoor Screening of Camelina sativa Germplasm.

In 2015, 420 accessions of Camelina sativa were requested from the USDA-National Plant Germplasm System, Plant Genetic Resources of Canada, the Plant Breeding and Acclimatization Institute (IHAR) based in Poland, and Dr. Johann Vollmann at the Universitat fir Bodenkultur Wien (BOKU), Vienna, Austria for agronomic characterization and winter hardiness evaluation. Each accession was planted in a single, 1.5 m row on Sep. 1, 2015 using a Wintersteiger Seedmech TRM 2200 plotmatic four-row cone planter with a 38 cm row spacing. Approximately 100 seeds were planted per row. On Nov. 25, 2015, each row was scored for the presence of reproductive stems or “bolting”. Accessions were scored as bolting or slow bolting.

Accessions that were scored as slow bolting were planted in triplicate in a randomized complete block design without vernalization in the greenhouse in St. Paul, MN on Feb. 19, 2016. The greenhouse temperature was maintained near 20° C. and was supplemented with halogen lighting to ensure 16 h light was available each day. The height of each plant was measured 34 days after planting.

On Sep. 13, 2017, a single four-row plot of each Camelina sativa accession that was previously identified as slow bolting winter types was planted, in addition to a single four-row plot of USDA-NPGS accession PI650163 (origin—Former Soviet Union) in a field in Rosemount, Minnesota, USA, in addition to multiple plots of the control accession, Ames 33292 (‘Joelle’). This experiment was planted as a seed increase. The seeding rate was 0.4 g per plot and a SRES 4-row plot planter was used with a 38 cm row spacing. The seeding depth was set to 1.27 cm; however, the actual seeding depth varied and was generally deeper due to uneven soil conditions.

On Oct. 18, 2018 a selected camelina line from accession PI 650163, referred to as PI 650163-1, and the winter camelina control accession, Ames 33292 (‘Joelle’), were each planted in a 1.5 m row in a field in St. Paul, Minnesota, USA. PI 650163-1 was identified in the aforementioned Rosemount, Minnesota field in May of 2018 as the earliest flowering plant present. The seeding rate was 0.1 g per row, the seeding depth was 1.27 cm and a SRES 4-row plot planter was used with a 38 cm row spacing.

On Sep. 9, 2019 camelina accessions PI 650163-1 and Ames 33292 were planted in duplicate in a field in St. Paul, Minnesota, in a completely randomized design to quantify differences in flowering time between these accessions. This site-year was planted by hand at a seeding rate of 0.1 g per 1.5 m row and a target seeding depth of 1.27 cm.

### Resequenced Camelina Accessions

Eleven camelina accessions selected for either winter hardiness, seed protein content, or early maturity were resequenced at the University of Minnesota (UMN) along with the 75 camelina accessions that were resequenced by Li et al., (2020) at Montana State University (MSU). The 11 UMN resequenced accessions were sequenced in three separate runs by the University of Minnesota Genomics Center with an Illumina NovaSeq 6000 platform on S1, S1, and S4 flow cells and yielded approximately 38×, 90×, and 80× coverage, respectively.

### Variant Calling Pipeline

For the Li et al., (2020) resequenced accessions, interleaved fastq.gz sequence files were downloaded from NCBI using Google Cloud Platform. Each interleaved fastq file was de-interleaved into two separate fastq files using a script provided by Daniel Standage on a Biostars forum post (https://www.biostars.org/p/141256/). Each of these files contained one of the two paired end read sequence results. These de-interleaved files were used for aligning to the Camelina sativa reference genome (Kagale et al., 2014) downloaded from the Crucifer Genome Initiative (developed by researchers in Dr. Isobel Parkin's lab as a collaboration between Agriculture and Agri-Food Canada and the Global Food Security Institute, http://cruciferseq.ca/). UMN data was released from the University of Minnesota Genomics Center as zipped, de-interleaved files. All sequence data was aligned using the Burrows-Wheeler transformation aligner (version 0.7.17-ri188, Li and Durbin, 2009)(bwa-mem), and Samtools (version 1.14, Danecek et al., 2021) was used for sorting and compressing the elements of each file. Variants were called using BCFtools (version 1.9, Danecek et al., 2021) and the utilities mpileup (using options -Ou -f) and call (using -Ou -my).

KASP Genotyping to Distinguish Functional from Subfunctional Alleles of FLC on Chromosome 20

DNA was isolated from a small leaf piece (˜1 mm×2 mm leaf sample) using Sigma-Aldrich® Extract-N-AMP DNA extraction (SKU: E7526) and dilution (SKU: D5688) solutions from MilliporeSigma Inc. (St. Louis, MO 63178). KASP primers were designed as a collaboration between us and Dr. Nisha Jain (3CR Bioscience, Enfield, England) and ordered from Integrated DNA Technologies, Inc (Table 1).

TABLE 1
Primer design for Kompetitive Allele Specific PCR (KASP) genotyping for
winter (allele X) versus spring (allele Y) growth habit in Camelina sativa
based upon the previously identified single basepair INDEL in exon 5 of the
FLOWERING LOCUS C gene on chromosome 20 (Anderson et al., 2018). The bold,
italic DNA sequence at the beginning of the X and Y KASP primers represent
the sequences that bind to the FAM and HEX fluorescent dyes, respectively.

| | |
|---|---|
| Cs_UMN_KASP_FLC | AATCAAATATCTAACCTTTTCTTTGAGGTT |
| 20_C (SEQ ID NO: 1) | |
| Cs_UMN_KASP_FLC | GAAGGTGACCAAGTTCATGCTTTTATTTGTTACAGACAGAAC |
| 20_X (SEQ ID NO: 2) | TAATGTT |
| Cs_UMN_KASP_FLC | GAAGGTCGGAGTCAACGGATTCTTTTATTTGTTACAGACAGA |
| 20_Y (SEQ ID NO: 3) | ACTAATGTG |

Tm and binding site affinity were estimated through SnapGene Viewer. The primer mix included 3.5 μL of the HEX/Y primer (fluorescence 533-580), 4.5 μL of the FAM/X primer (fluorescence 465-510), 13 μL of the common primer, and 20 μL of nuclease free water. The PCR master mix used was 3CR Bioscience's PACE2.0 Genotyping Master Mix 2× low ROX (003-0006), and was combined with the primer mix at the following rate per reaction: 0.15 μL primer mix, 5 μL PACE2.0 Master Mix, 5 μL nuclease free water and 1 μL of DNA. KASP genotyping PCR settings on a Roche LightCycler® 480 were:
1. 95° C. enzyme activation for 15 minutes
2. A touchdown temperature cycle starting with an annealing temperature of 59° C. for one minute, then a melting temperature of 95° C. for 10 seconds, with a step down of the annealing temperature of 2.2° C. until an annealing temperature of 51° C. was reached.
3. An annealing temperature of 50° C. was used for the remainder of the PCR—all other temperatures and durations remained the same.

### Bulked Segregant Analysis for Flowering Time

From an F₂ population of 75 plants segregating for flowering time from a cross of PI 650163-1 and Joelle), seven early flowering plants and eight late flowering plants were selected to form DNA pools for bulked segregant analysis. DNA was first isolated from each plant individually using a Qiagen DNeasy® Plant Mini Kit, then an equal amount of DNA from each early flowering plant (180 ng) was pooled and likewise for each late flowering plant (250 ng). This DNA was submitted to the University of Minnesota Genomics Center for whole genome sequencing using NovaSeq on a S4 flow cell to achieve a target coverage of 80×.

### Results and Discussion

Discovery of a Group of Spring Camelina Accessions that are Homozygous for Functional FLC Alleles on Chromosome 20

In Arabidopsis, the FLC gene is required for the winter growth habit. The FLC gene in Arabidopsis codes for a protein that has been shown to suppress the flowering promoter gene, FLOWERING LOCUS T (FT) (Samach et al., 2000), and is itself suppressed (methylated) by proteins that are coded by genes activated by exposure to cool temperatures (i.e. 5° C.±1° C.) (Sheldon et al., 2000). Polymorphisms in orthologous FLC alleles can differentiate the spring versus winter growth habit. In the winter camelina accession Joelle (Ames 33292), FLC is expressed on chromosomes 8 and 20, but not on chromosome 13, while its only expressed on chromosome 8 in the spring camelina accession ‘CO46’ (Anderson et al., 2018). The subfunctional allele of this gene in spring type camelina has a frameshift mutation in exon 5 on chromosome 20 at bp position 4,195,043 (Anderson et al., 2018). Analysis of these alleles more broadly among a greater number of accessions within the camelina germplasm phenotyped for growth habit and resequenced by Li et al. (2020) (with nearly identical phenotypes here) revealed that there are at least 20 exceptional cases in which a spring accession is homozygous for the winter FLC allele on chromosome 20, two cases in which a spring accession is homozygous and three cases in which a spring accession is heterozygous for the winter FLC allele on chromosome 8, and two cases in which a winter accession is homozygous for the spring FLC allele on chromosome 8 (Table 2). For the cases that involved a heterozygous genotype where homozygosity is expected in this self-pollinating species, without wishing to be bound by theory, possible explanations are that either there was a rare case of outcrossing or resequencing sample contamination.

TABLE 2
Camelina sativa accessions that were resequenced and analyzed for FLOWERING LOCUS
C alleles that could predict growth habit. Resequenced by either the University of
Minnesota or by Montana State University. Spring growth habit plants with winter alleles
or winter growth habit plants with spring alleles are marked with an asterisk

| | | | | | FLC8 | FLC20 |
|---|---|---|---|---|---|---|
| Accession | Accession ID Li | | | Growth | Winter | Winter |
| ID | et al. (2020) | Accession Name | NCBI Run | Habit | Allele | Allele |
| PI 650163 | CS229UMN | CSS-CAM37 | UMN | Facultative | het | |
| | | | Resequenced | winter | | |
| PI 650163-1 | CS229-1UMN | CSS-CAM37 | UMN | Facultative | | |
| | | | Resequenced | winter | | |
| PI 650163 | CS229 | CSS-CAM37 | SRR9993039 | Facultative | | |
| | | | | winter | | |
| PI 597833 | CS201 | 163-2073-72 | SRR9987775 | Spring | | |
| PI 652885 | CS235 | Ames 29309 | SRR10028112 | Spring | | |
| PI 652886 | CS236 | Ames 29310 | SRR10028114 | Spring | | |
| CN 113755 | CS110UMN | Bavaria | UMN | Spring | | x |
| | | | Resequenced | | | |
| CAM 180 | CS110 | Bavaria | SRR9914681 | Spring | | x |
| CAM 61 | CS030 | Boha1 | SRR9722050 | Spring | | |
| PI 650144 | CS210 | Boha2 | SRR10873755 | Spring | | |
| PI 311735 | CS199 | Borowska | SRR10873743 | Spring | | x |
| CAM 223 | CS142 | Borowska-ihar | SRR9924268 | Spring | | |
| CAM 108 | CS063 | Bronowska | SRR9722689 | Spring | | x |
| IHAR | CS211_IHAR_UMN | BRSCHW 28347 | UMN | Spring | | |
| 247026 | | | Resequenced | | | |
| PI 650145 | CS211 | BRSCHW-28347 | SRR9990156 | Spring | | x |
| PI 650146 | CS212 | BRSCHW-30021 | SRR9990305 | Spring | | |
| CAM 63 | CS031 | CAME1 | SRR9722051 | Spring | | |
| PI 650140 | CS206 | Came3 | SRR9989930 | Spring | | |
| PI 650147 | CS213 | Came4 | SRR9990304 | Spring | | |
| PI 650152 | CS218 | CPS-CAM23 | SRR10027314 | Spring | | |
| PI 650153 | CS219 | CPS-CAM10 | SRR9990527 | Spring | x | |
| PI 633192 | CS202 | CR-476/65 | SRR9988371 | Spring | | x |
| PI 633193 | CS203 | CR-492/94a | SRR9988367 | Spring | | x |
| PI 650161 | CS227 | CSS_CAM35 | SRR9992927 | Spring | | x |
| PI 650154 | CS220 | CSS-CAM25 | SRR10027318 | Spring | | |
| PI 650156 | CS222 | CSS-CAM29 | SRR9990625 | Spring | | |
| PI 650157B | CS223 | CSS-CAM30 | SRR9990812 | Spring | | |
| PI 650159 | CS225 | CSS-CAM33 | SRR9992827 | Spring | | |
| PI 650160 | CS226 | CSS-CAM34 | SRR9992839 | Spring | | |
| PI 650162 | CS228 | CSS-CAM36 | SRR9993037 | Spring | | |
| PI 650164 | CS230 | CSS-CAM38 | SRR10027313 | Spring | | x |
| PI 650165 | CS231 | CSS-CAM7 | SRR10001240 | Spring | | |
| PI 650166 | CS232 | CSS-CAM8 | SRR10027259 | Spring | | |
| CAM 74 or | CS039 or 071 | Czenstochwska- | SRR9722741 | Spring | | |
| 123 | | | | | | |
| Ames 31231 | CS191 | GE.2011-01 | SRR9983193 | Spring | | |
| Ames 31232 | CS192 | GE.2011-05 | SRR9983388 | Spring | | x |
| PI 650148 | CS214 | Giessen3 | SRR9990306 | Spring | | |
| PI 650149 | CS215 | Giessen4 | SRR9990308 | Spring | | x |
| PI 633194 | CS204 | GiessenNr.-3 | SRR9988728 | Spring | | x |
| CAM 171 | CS105 | Guillemeteau | SRR9905074 | Spring | | x |
| CAM 67 | CS034 | Hoga1 | SRR9722216 | Spring | | |
| PI 650150 | CS216 | Hoga2 | SRR9990309 | Spring | | |
| PI650167B | CS233 | Index Seminum 144 | SRR10027319 | Spring | | |
| CN 113753 | CS107UMN | Irkutskaja-mestnaja | UMN | Spring | | x |
| | | | Resequenced | | | |
| CAM 173 | CS107 | Irkutskaja-mestnaja | SRR10873422 | Spring | | x |
| | UMN | | UMN | | x | |
| Vavilov 407 | | Karelia | Resequenced | Spring | | |
| CAM 7 or 57 | CS003 or 027 | Kirgizskij | SRR9721948 | Spring | | |
| CAM 40 | CS020 | Lindo | SRR9721882 | Spring | | |
| PI 304268 | CS195 | No. 401 | SRR9986031 | Spring | | |
| PI 304269 | CS196 | No. 402 | SRR9987777 | Spring | | |
| PI 650141 | CS207 | NU-52279 | SRR9989959 | Spring | | |
| ? | ? | Omskij | SRR10873371 | Spring | | |
| CAM 25 | CS007 | Omskij-Mestnyi | SRR9721935 | Spring | | x |
| CAM 99 | CS059 | PRFGL.10 | SRR9722541 | Spring | | |
| CAM 84 | CS047 | PRFGL.42 | SRR9722430 | Spring | | |
| CAM 90 | CS053 | PRFGL.54 | SRR9722466 | Spring | | |
| MT102 | CS251 | Przyth | SRR9721197 | Spring | | |
| CAM 165 | CS099 | STAMM04X05 | SRR9733108 | Spring | | |
| CAM 196 | CS118 | STAMM06X10B | SRR9915684 | Spring | | |
| CAM 257 | CS172 | STAMM06X11B | SRR9974986 | Spring | | |
| CAM 151 | CS085 | STAMM07X13B | SRR9727008 | Spring | | |
| CAM 157 | CS091 | STAMM10X12 | SRR9730513 | Spring | | x |
| MT 5 | CS001 | Suneson | SRR9721195 | Spring | | x |
| PI 650151 | CS217 | Svalof-AM_CSA217 | SRR9990325 | Spring | | |
| CAM 68 | CS035 | Svalof-AM_CSA35 | SRR9722218 | Spring | | |
| CAM 35 | CS015 | Ukrajinskij | SRR9721670 | Spring | | x |
| PI 258366 | CS193 | Vniimk-17 | SRR9983359 | Spring | | x |
| CAM 110 | CS064 | Volynskaja | SRR9722690 | Spring | | |
| PI 258367 | CS194 | Voronezh-349 | SRR9986045 | Spring | | |
| ? | ? | Voronezskij | SRR10873332 | Spring | x | |
| CAM 221 | CS141 | Voronezskij 3392 | SRR10873735 | Spring | het | |
| CAM 174 | CS108 | Voronezskij 3491 | SRR9914678 | Spring | | x |
| CAM 120 | CS070 | Wroclawska | SRR9722738 | Spring | | x |
| CAM 78 | CS041 | Zarja socialisma- | SRR9722315 | Spring | het | het |
| | | Auslese-1 | | | | |
| *PI 650143 | CS209 | CS-CROO | SRR9990157 | Winter | | x |
| PI 650155 | CS221 | CSS-CAM27 | SRR9990588 | Winter | x | x |
| PI 650158 | CS224UMN | CSS-CAM31 | UMN | Winter | x | x |
| | | | Resequenced | | | |
| PI 650168 | CS234UMN | NE2006-1 | UMN | Winter | x | x |
| | | | Resequenced | | | |
| PI 650168 | CS234 | NE2006-1 | SRR10028113 | Winter | x | x |
| PI 311736 | CS200 | Przybrodzka | SRR 10001246 | Winter | x | x |
| IHAR | CS200-IHAR- | Przybrodzka | UMN | Winter | x | x |
| 502792 | UMN | | Resequenced | | | |
| CAM 30 | CS010 | Zarja socialisma1 | SRR9721666 | Winter | no call | x |
| CAM 172 | CS106 | Zarja socialisma2 | SRR10873423 | Winter | x | x |
| CN 113691 | CS040UMN | Zarja socialisma- | UMN | Winter | x | x |
| | | Auslese-2 | Resequenced | | | |
| CAM 705 | CS040 | Zarja socialisma- | SRR9722307 | Winter | x | x |
| | | Auslese-2 | | | | |
| Ames 33292 | UMN | Joelle | UMN | Winter | x | x |
| | | | Resequenced | | | |

### Bulked Segregant Analysis for Flowering Time

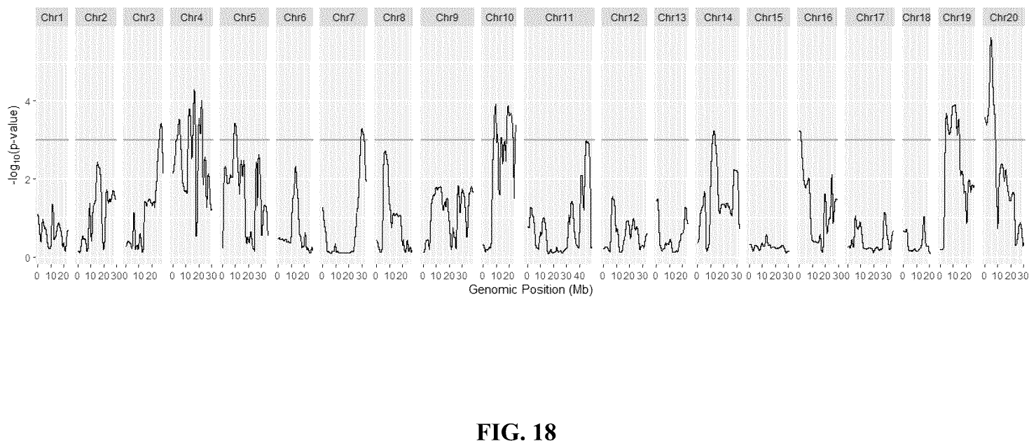

The objective of this experiment was to identify the primary chromosome specific gene(s) controlling flowering time differences between the facultative winter accession of camelina PI 650163-1 and a winter accession Ames 33292 (Joelle). The QTLseqr R-package analysis identified just one major peak of QTL LOD score values (−log 10 of variant p-values) in the bulked segregant analysis experiment (FIG. 18). The variant (SNP) with the maximum LOD score (5.6) was at position 4.7 Mb on chromosome 20, was in an intergenic region and thus was unlikely to influence the flowering time trait. However, it was likely in linkage with the frameshift inducing INDEL in FLC on chromosome 20 (Anderson et al., 2018), which had a LOD score of 5.4 and is at position 4.2 MB. This result supports the theory that FLC on chromosome 20 is the primary regulator of flowering time, rather than FLC on chromosome 8 (for which PI 650163-1 has a subfunctional allele) or any other regulatory genes related to flowering time for PI 650163-1. There may have been a few smaller signals of SNP effects on chromosomes 4, 10, and 19, but none were comparable to the signal on chromosome 20. Though the fully functional allele of FLC on chromosome 8 is very conserved and highly expressed among winter accessions and the subfunctional allele is conserved among spring accessions (Anderson et al., 2018), it appears that it plays a smaller role in controlling flowering time on the growth habit level in camelina, despite the fact that another report showed it has a significant effect on flowering in spring camelina backgrounds (Li et al., 2020).

### KASP Genotyping Approach Distinguished Between Functional and Subfunctional Alleles of FLC on Chromosome 20

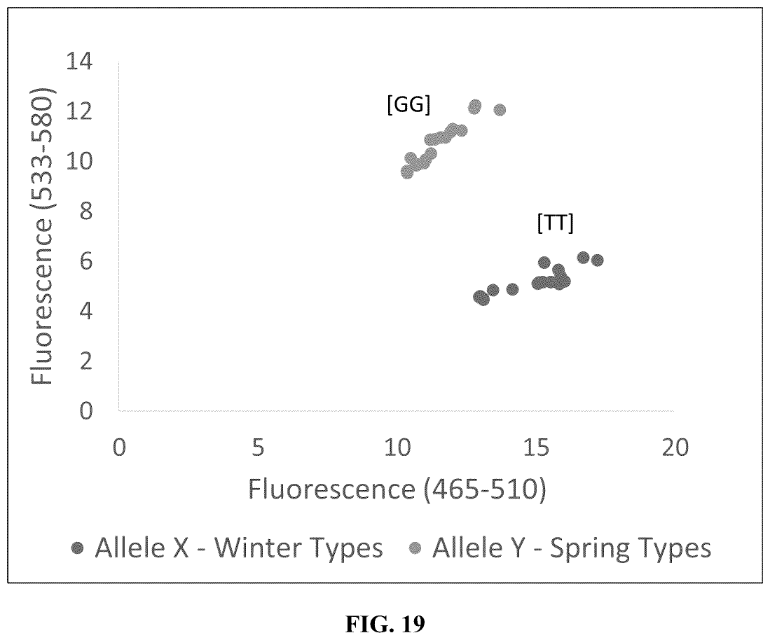

Currently, the only genotyping approach to distinguish functional from subfunctional alleles of the FLC gene on chromosome 20 is qRT-PCR, which was developed from camelina accessions shared through an intermediary (Chao et al., 2019). Many plant breeding programs make use of DNA-based Kompetitive Allele Specific PCR (KASP) genotyping. Thus, this genotyping approach was selected to be optimized for this marker. KASP primers were designed for the SNV found in exon 5 of FLC on chromosome 20. While individual primers in this primer set contained many off target binding sites, these sites were not adjacent to each other and thus would not allow for off target amplification to a degree that could confound results. This KASP primer set was effective in distinguishing winter from spring type camelina. (FIG. 19).

### Discovery of Facultative Winter Camelina Accession PI650163

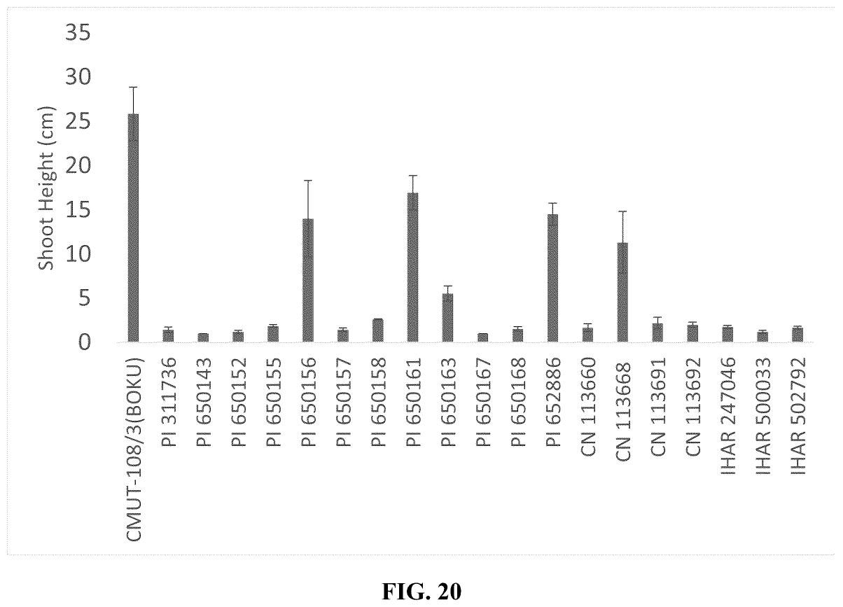

The majority of the accessions within the tested camelina germplasm had a spring-type growth habit. Based upon previous observations of bolting in the fall of 2015, a follow-up greenhouse experiment was designed and conducted in which each accession/line (20) that was scored as slow bolting was planted into pots in triplicate without a vernalizing cold treatment at any point. Fourteen of these accessions did not bolt within the time frame of the experiment. PI 650163 repeated the slow bolting phenotype observed in fall 2015, four bolted at a moderate rate, and one bolted very quickly (FIG. 20). The latter may have been subjected to a more stressful micro-environment in fall 2015 resulting in it being mislabeled as slow bolting. By spring of 2016, none of the spring type camelina plants had survived the Minnesota winter.

### Discovery of Winter-Hardy, Early Flowering and Maturing Camelina Accession PI 650163-1

In the fall of 2017, PI650163 was planted in Rosemount, Minnesota, USA, with the hypothesis that it may be able to survive a Minnesota winter due to its facultative (slow bolting) winter type growth habit. On May 18, 2018, a single plant from this plot (PI 650163-1) was selected, since it was the first plant flowering in this plot as well as in the entire field. PI 650163-1 flowered nine and 17 days before Ames 33292 (‘Joelle’) in 2019 and 2020 in St. Paul, MN, respectively (Table 3). Physiological maturity was also scored in St. Paul, MN in 2020, using the BBCH scale developed by Martinelli and Galasso (2011). PI 650163-1 reached 50% maturity nine days before Ames 33292 (Table 3).

TABLE 3
Flowering and maturity visual observations made in the field
in St. Paul. Single 1.5 m rows of Camelina were planted on Oct. 18^{th},
2018 (n = 1) and Sep. 9^{th}, 2019 (n = 3), respectively.

| | 2019 | 2020 | | | |
|---|---|---|---|---|---|
| | First Plant | 50% | | 50% Phys. | |
| Accession | Flowered | Flowered | SE | Maturity | SE |
| Ames 33292 | 25-May | 18.5-May | 1.5 | 27-Jun | 1 |
| (‘Joelle’) | | | | | |
| PI 650163-1 | 16-May | 1.5-May | 1.5 | 16-Jun | 1 |

In canola, an example of the genetic basis of the facultative winter type is increased expression of a repressor of FLC—the VENALIZATION INSENSITIVE 3 (VIN3) gene, which is typically upregulated after exposure to temperatures near 4° C. In facultative winter type canola, VIN3 has increased expression even at a temperature of 20° C., far above what typically induces its expression of this gene, and thus baseline FLC expression is relatively lower in facultative winter type canola than in winter type (Huang et al., 2021). Camelina sativa accession PI 650163 and PI 650163-1 did not have any obvious variants in VIN3, but one or more other genes in this pathway may have an allele influencing this phenotype. However, since these facultative winter accessions already have a subfunctional allele of FLC on chromosome 20, this floral suppression pathway may be less likely to influence this phenotype. Subfunctional genes in the floral promoter pathway such as FLOWERING LOCUS T (FT) and CONSTANS (CO) could also be influencing this phenotype, but again, these accessions did not have any obvious variants in FT or in CO.

Without wishing to be bound by theory, the new approach developed in the work presented herein using KASP genotyping to differentiate functional versus subfunctional FLC alleles on chromosome 20 is expected to accelerate camelina breeding efforts by making genotyping available on widely used platforms and minimizing costs.

The facultative winter camelina accession identified herein—PI 650163-1—appears to hold promise for cropping systems in the Upper Midwest, since it has demonstrated robust winter hardiness in the site years of this study, but also flowers and matures earlier, allowing the primary summer annual crops to receive full light interception sooner.

### REFERENCES

Anderson, J. V, Horvath, D. P., Dogramaci, M., Dom, K. M., Chao, W. S., Watkin, E. E., Hernandez, A. G., Marks, M. D., Gesch, R., 2018. Expression of FLOWERING LOCUS C and a frameshift mutation of this gene on chromosome 20 differentiate a summer and winter annual biotype of Camelina sativa. Plant direct 2, e00060. https://doi.org/10.1002/pld3.60
Brock, J. R., Donmez, A. A., Beistein, M. A., Olsen, K. M., 2018. Phylogenetics of Camelina Crantz. (Brassicaceae) and insights on the origin of gold-of-pleasure (Camelina sativa). Mol. Phylogenet. Evol. 127, 834-842. https://doi.org/10.1016/j.ympev.2018.06.031
Chao, W. S., Wang, H., Horvath, D. P., Anderson, J. V, 2019. Selection of endogenous reference genes for qRT-PCR analysis in Camelina sativa and identification of FLOWERING LOCUS C allele-specific markers to differentiate summer- and winter-biotypes. Ind. Crops Prod. 129, 495-502. https://doi.org/10.1016/j.indcrop.2018.12.017
Chen H, Wang T, He X, Cai X, Lin R, Liang J, Wu J, King G, Wang X. 2022. [WWW Document] http://brassicadb.cn/#. Brassicaceae Database (BRAD). Nucleic Acids Res. 50 (D1). D1432-D1441. (accessed 3.10.2022).
Crucifer Genome Initiative, n.d. Agriculture and Agri-Food Canada Saskatoon Research and Development Center in collaboration with the Global Institute for Food Security and Dr. Isobel Parkin's lab. [WWW Document].
Danecek, P., Bonfield, J. K., Liddle, J., Marshall, J., Ohan, V., Pollard, M. O., Whitwham, A., Keane, T., McCarthy, S. A., Davies, R. M., Li, H., 2021. Twelve years of SAMtools and BCFtools. Gigascience 10. https://doi.org/10.1093/gigascience/giab008
Eberle, C. A., Thom, M. D., Nemec, K. T., Forcella, F., Lundgren, J. G., Gesch, R. W., Riedell, W. E., Papiernik, S. K., Wagner, A., Peterson, D. H., Eklund, J. J., 2015. Using pennycress, camelina, and canola cash cover crops to provision pollinators. Ind. Crops Prod. 75, 20-25. https://doi.org/10.1016/j.indcrop.2015.06.026
Gesch, R. W., Cermak, S. C., 2011. Sowing date and tillage effects on fall-seeded camelina in the northern corn belt. Agron. J. 103, 980-987. https://doi.org/10.2134/agronj2010.0485
Hotton, S. K., Kammerzell, M., Chan, R., Hernandez, B. T., Young, H. A., Tobias, C., McKeon, T., Brichta, J., Thomson, N.J., Thomson, J. G., 2020. Phenotypic examination of Camelina sativa (L.) Crantz accessions from the USDA-ARS national genetics resource program. Plants 9, 642. https://doi.org/10.3390/plants9050642
Huang, C. H., Sun, R. R., Hu, Y., Zeng, L. P., Zhang, N., Cai, L. M., Zhang, Q., Koch, M. A., Al-Shehbaz, I., Edger, P. P., Pires, J. C., Tan, D. Y., Zhong, Y., Ma, H., 2016. Resolution of Brassicaceae Phylogeny Using Nuclear Genes Uncovers Nested Radiations and Supports Convergent Morphological Evolution. Mol. Biol. Evol. 33, 394-412. https://doi.org/10.1093/molbev/msv226
Huang, L. Y., Min, Y., Schiessl, S., Xiong, X. H., Jan, H. U., He, X., Qian, W., Guan, C. Y., Snowdon, R. J., Hua, W., Guan, M., Qian, L. W., 2021. Integrative analysis of GWAS and transcriptome to reveal novel loci regulation flowering time in semi-winter rapeseed. PLANT Sci. 310. https://doi.org/10.1016/j.plantsci.2021.110980
Kagale, S., Koh, C. S., Nixon, J., Bollina, V., Clarke, W. E., Tuteja, R., Spillane, C., Robinson, S. J., Links, M. G., Clarke, C., Higgins, E. E., Huebert, T., Sharpe, A. G., Parkin, I. A. P., 2014. The emerging biofuel crop Camelina sativa retains a highly undifferentiated hexaploid genome structure. Nat. Commun. 5. https://doi.org/10.1038/ncomms4706
Li, H., Durbin, R., 2009. Fast and accurate short read alignment with Burrows-Wheeler transform. BIOINFORMATICS 25, 1754-1760. https://doi.org/10.1093/bioinformatics/btp324
Li, H., Hu, X., Lovell, J. T., Grabowski, P. P., Mamidi, S., Chen, C., Amirebrahimi, M., Kahanda, I., Mumey, B., Barry, K., Kudrna, D., Schmutz, J., Lachowiec, J., Lu, C. F., 2021. Genetic dissection of natural variation in oilseed traits of camelina by whole-genome resequencing and QTL mapping. Plant Genome 14. https://doi.org/10.1002/tpg2.20110
Madeira, F., Pearce, M., Tivey, A. R. N., Basutkar, P., Lee, J., Edbali, O., Madhusoodanan, N., Kolesnikov, A., Lopez, R., n.d. Search and sequence analysis tools services from EMBL-EBI in 2022. Nucleic Acids Res. https://doi.org/10.1093/nar/gkac240
Martinelli, T., Galasso, I., 2011. Phenological growth stages of Camelina sativa according to the extended BBCH scale. Ann. Appl. Biol. 158, 87-94. https://doi.org/10.1111/j.1744-7348.2010.00444.x
Ott, M. A., Eberle, C. A., Thom, M. D., Archer, D. W., Forcella, F., Gesch, R. W., Wyse, D. L., 2019. Economics and Agronomics of Relay-Cropping Pennycress and Camelina with Soybean in Minnesota. Agron. J. 111, 1281-1292. https://doi.org/10.2134/agronj2018.04.0277
Samach, A., Onouchi, H., Gold, S. E., Ditta, G. S., Schwarz-Sommer, Z., Yanofsky, M. F., Coupland, G., 2000. Distinct roles of CONSTANS target genes in reproductive development of Arabidopsis. Science (80). 288, 1613-1616. https://doi.org/10.1126/science.288.5471.1613
Sheldon, C. C., Rouse, D. T., Finnegan, E. J., Peacock, W. J., Dennis, E. S., 2000. The molecular basis of vernalization: The central role of FLOWERING LOCUS C (FLC). Proc. Natl. Acad. Sci. U.S.A 97, 3753-3758. https://doi.org/10.1073/pnas.060023597
Standage, D., 2015. deinterleave fastq file [WWW Document]. Biostars, Bioinforma. Explained.
Sustainable Oils Camelina [WWW Document], n.d. URL https://susoils.com/. (accessed 2.13.22).
Weyers, S., Thom, M., Forcella, F., Eberle, C., Matthees, H., Gesch, R., Ott, M., Feyereisen, G., Strock, J., Wyse, D., 2019. Reduced Potential for Nitrogen Loss in Cover Crop-Soybean Relay Systems in a Cold Climate. J. Environ. Qual. 48. https://doi.org/10.2134/jeq2018.09.0350
Wittenberg, A., Anderson, J. V, Berti, M. T., 2020. Crop growth and productivity of winter camelina in response to sowing date in the northwestern Corn Belt of the USA. Ind. Crops Prod. 158, 113036. https://doi.org/10.1016/j.indcrop.2020.113036
Zohary, D., Hopf, M., Weiss, E., 2012. Domestication of Plants in the Old World: The Origin and Spread of Domesticated Plants in Southwest Asia, Europe, and the Mediterranean Basin. OUP Oxford.

### DEPOSIT INFORMATION

Camelina sativa Variety ‘CMN2207’

A deposit of at least 625 seeds of the Camelina sativa variety ‘CMN2207’ was made with the American Type Culture Collection (ATCC), ATCC Patent Depository, 10801 University Boulevard, Manassas, Virginia, 20110, USA, and assigned ATCC number X1. The seeds deposited with the ATCC Patent Depository on DATE were obtained from the seed of the variety maintained by the Regents of the University of Minnesota, 200 Oak Street SE, Suite 280, Minneapolis, MN, 55455, since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the Camelina sativa variety ‘CMN2207’ will be maintained in the ATCC Patent Depository, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

What is claimed is:

1. A Camelina sativa seed designated as ‘CMN2207’, a representative sample of seed having been deposited under ATCC Accession Number X1.

2. A Camelina sativa plant produced by growing the seed of claim 1.

3. A plant part from the plant of claim 2, wherein said part is a leaf, a flower, an ovule, a pollen grain, a seed pod, a seed, a fruit, a cell, or a portion thereof.

4. The plant part of claim 3, wherein said part is a seed.

5. A Camelina sativa plant having all the physiological and morphological characteristics of the Camelina sativa plant of claim 2.

6. A plant part from the plant of claim 5, wherein said part is a leaf, an ovule, a pollen grain, a seed pod, a seed, a fruit, a cell, or a portion thereof.

7. The plant part of claim 6, wherein said part is a seed.

8. An F1 hybrid Camelina sativa plant having ‘CMN2207’ as a parent where ‘CMN2207’ is grown from the seed of claim 1.

9. A pollen grain or an ovule of the plant of claim 2.

10. A tissue or cell culture produced from protoplasts or cells from the plant of claim 2, wherein said cells or protoplasts are produced from a plant part selected from the group consisting of root, root tip, meristematic cell, stem, hypocotyl, petiole, cotyledon, leaf, flower, anther, pollen, pistil, embryo, seed, and fruit

11. A Camelina sativa plant regenerated from the tissue culture of claim 10, wherein the plant has all of the morphological and physiological characteristics of a Camelina sativa plant produced by growing Camelina sativa seed designated as ‘CMN2207’, representative sample of seed having been deposited under ATCC Accession Number X1.

12. A method of making Camelina sativa seeds, the method comprising crossing the plant of claim 2 with itself or with another Camelina sativa plant and harvesting seed therefrom.

13. The method of claim 12, wherein the method further comprises:
(a) selecting one or more of the F1 progeny for plants that have at least one desired trait to produce selected progeny plants;
(b) crossing the selected progeny plants with ‘CMN2207’ to produce backcross progeny plants, wherein a representative sample of seed of ‘CMN2207’ having been deposited under ATCC Accession Number X1;
(c) selecting for backcross progeny plants that have the desired trait and all of the physiological and morphological characteristics of ‘CMN2207’ when grown in the same environmental conditions to produce selected backcross progeny plants; and
(d) repeating steps (b) and (c) three or more times in succession to produce selected fourth or higher backcross progeny plants that comprise the desired trait and all of the physiological and morphological characteristics of ‘CMN2207’ when grown in the same environmental conditions.

14. The method of claim 13, wherein the at least one desired trait is selected from the group consisting of: ability to flower absent vernalization; winter hardiness; reaching maturity earlier than control fully winter Camelina accessions when grown under the same conditions; resistance to Ceutorhynchus erysimi (weevils); seed area; seed mass; seed yield; and plant height.

15. The method of claim 14, wherein a plant is deemed to mature earlier than the control plant when a plot of said plant reaches 50% maturity in fewer days than a plot of the control plant.

16. A method of producing a seed of ‘CMN2207’-derived Camelina sativa plant, comprising:
a) crossing a Camelina sativa plant designated as ‘CMN2207’ with a second Camelina sativa plant, whereby seed of a ‘CMN2207’-derived Camelina sativa plant forms.

17. The method of claim 16, further comprising:
b) crossing a plant grown from ‘CMN2207’-derived Camelina sativa seed with itself or a second Camelina sativa plant to yield additional ‘CMN2207’-derived Camelina sativa seed;
c) growing the additional ‘CMN2207’-derived Camelina sativa seed of step (b) to yield additional ‘CMN2207’-derived Camelina sativa plants; and
d) repeating the crossing and growing of steps (b) and (c) for an additional 3-10 generations to generate further ‘CMN2207’-derived Camelina sativa plants.

18. A method of producing a Camelina sativa seed, said method comprising growing the plant of claim 2 until it produces at least one seed pod, and harvesting the seeds.

19. A method of producing a seed of ‘CMN2207’-derived Camelina sativa, comprising:
a) crossing a Camelina sativa plant designated as ‘CMN2207’, representative sample of seed having been deposited under ATCC Accession Number X1, with itself or with a second Camelina sativa plant, whereby seed of a ‘CMN2207’-derived Camelina sativa plant forms.

20. The method of claim 19 further comprising:
b) crossing a plant grown from ‘CMN2207’-derived Camelina sativa seed with itself or with a second Camelina sativa plant to yield additional ‘CMN2207’-derived Camelina sativa seed;
c) growing the additional ‘CMN2207’-derived Camelina sativa seed of step (b) to yield additional ‘CMN2207’-derived Camelina sativa plants; and
d) repeating the crossing and growing of steps (b) and (c) for an additional 3-10 generations to generate further ‘CMN2207’-derived Camelina sativa plants.

21. A method of vegetatively propagating a plant of Camelina sativa ‘CMN2207’, the method comprising the steps of: (a) collecting tissue capable of being propagated from a plant of Camelina sativa ‘CMN2207’, representative seed of said Camelina sativa variety having been deposited under ATCC Accession Number X1; and (b) producing a rooted plant from said tissue.

22. A Camelina sativa plant produced by the method of any one of claims 12-21, wherein said plant reaches maturity earlier than control fully winter Camelina accessions when grown under the same conditions, wherein said plant flowers absent vernalization, and wherein said plant comprises a subfunctional FLC allele on chromosome 20.

23. A plant part from the plant of claim 22, wherein the part is a leaf, an ovule, a pollen grain, a seed pod, a seed, a fruit, a cell, or a portion thereof.

24. A Camelina sativa plant, wherein said Camelina sativa plant 1) flowers and reaches maturity earlier than a control winter Camelina accession when grown under the same conditions, 2) flowers and reaches maturity later than a control spring Camelina accession when grown under the same conditions without vernalization in a non-freezing environment, 3) can survive in a freezing environment after transitioning to a floral meristem, and 4) is able to flower absent vernalization.

25. The Camelina sativa plant of claim 24, wherein said plant further exhibits at least one trait selected from the group consisting of: resistance to Ceutorhynchus erysimi (weevils); a seed area in the range of about 1.2 mm² to about 1.4 mm²; a seed mass in the range of about 0.7 grams per thousand seeds to about 1 gram per thousand seeds; a seed yield in the range of about 1100 kg/ha to about 1300 kh/ha; and a plant height in the range of about 57 cm to about 65 cm.

26. The Camelina sativa plant of claim 24, wherein said Camelina sativa plant comprises a non-functional FLOWERING LOCUS C gene on chromosome 20.

27. The Camelina sativa plant of claim 24, wherein representative seeds of such plant have been deposited under ATCC Accession Number X1.


SEQUENCE LISTING (3 sequences)

SEQ ID NO: 1 - DNA, 30 bp, synthetic construct
  source 1..30: mol_type = other DNA
aatcaaatatctaaccttttctttgaggtt

SEQ ID NO: 2 - DNA, 49 bp, synthetic construct
  source 1..49: mol_type = other DNA
gaaggtgaccaagttcatgcttttatttgttacagacagaactaatgtt

SEQ ID NO: 3 - DNA, 51 bp, synthetic construct
  source 1..51: mol_type = other DNA
gaaggtcggagtcaacggattcttttatttgttacagacagaactaatgtg